(19) United States
(12) Patent Application Publication
(10) Pub. No.: US 2023/0364302 A1
(43) Pub. Date: Nov. 16, 2023

(54) CELLULOSE SCAFFOLDS DERIVED FROM MACROALGAE, PROCESS FOR THE PREPARATION THEREOF AND USES THEREOF

(71) Applicant: Ramot at Tel-Aviv University Ltd., Tel Aviv (IL)

(72) Inventors: Nurit Bar-Shai, Tel Aviv (IL); Alexander Golberg, Tel Aviv (IL); Ayelet Lesman, Tel Aviv (IL); Orna Sharabani-Yosef, Tel Aviv (IL)

(21) Appl. No.: 18/315,891

(22) Filed: May 11, 2023

Related U.S. Application Data
(60) Provisional application No. 63/340,579, filed on May 11, 2022.

(51) Int. Cl. A61L 27/36

(52) U.S. Cl. CPC A61L 27/3637

(57) ABSTRACT

Cellulose-based scaffolds having fibrous structures, which include a decellularized macroalgae tissue from which cellular materials and nucleic acids are removed; implants including such cellulose-based scaffolds; and a decellularization process for the preparation thereof. The macroalgae tissue may be a green macroalgae tissue, a red macroalgae tissue, or a brown macroalgae tissue. The green macroalgae tissue may be a Cladophora sp. tissue; and the red macroalgae tissue may be a Bangia sp. tissue.

### PRIORITY AND CROSS REFERENCE TO RELATED APPLICATIONS

The present application claims the benefit of U.S. Provisional Application No. 63/340,579, filed May 11, 2022, the entire content of which being herewith incorporated by reference as if fully disclosed herein.

### TECHNICAL FIELD

The present invention relates to a cellulose-based scaffold having a fibrous structure, which comprises a decellularized macroalgae tissue from which cellular materials and nucleic acids are removed; an implant comprising such a cellulose-based scaffold; and a decellularization process for the preparation thereof.

### BACKGROUND ART

In native tissues, the extracellular matrix (ECM) is an essential platform that fulfills several functions, including providing structural support for cell growth, impact on cell behavior, and stimulating tissue regeneration. Many of the challenges we face today are concerned with designing cost effective and safe alternatives to the technologies and materials currently in use to create microenvironments that would mimic the biochemical and physiological structures of natural environments within the human body. Multiple fabrication strategies and material sources have been investigated as promising biomaterials with novel properties. Among natural materials that serve as scaffolds for tissue engineering, cellulose-based matrices are relatively new to this research field, and are currently being investigated to facilitate mammalian cell culture in vitro and in vivo (Hickey and Pelling, 2019).

Cellulose is the most abundant polymer in nature and is a key structural element of the cell wall of plants, which gives the cell its mechanical strength and rigidity. In cotton, for example, it accounts for about 90% of the plant cell wall content. Together with lignin and hemicellulose, it supports plant's vertical growth. It is a stable polymer that consists of tightly packaged glucose monomers, which provides it with a highly organized structure, difficult to break apart and unfavorable to biodegradation in the absence of cellulolytic enzymes (Gibson, 2012). These characteristics give cellulose unique biophysical and biomechanical properties, which are stable over a long time. As such, it can preserve its shape with minimal deformation, and function as a permanent construct or as a structural support, that could ideally be used as a template to guide the restructuring of cells and newly formed tissue for various applications, such as skin and wound dressings, bone tissue, blood vessels, neural, muscle, tendons, cartilage, vertebrae disks, urinary tracts, and larynx tissues, to name a few (Hickey and Pelling, 2019). Moreover, cellulose hydrophilicity and fluid uptake could provide a moist construct for wound healing environment, while promoting interaction with negatively charged cell surface, and thus advancing cell adhesion and proliferation (Hickey and Pelling, 2019). Cellulose sources in tissue engineering range from natural polymers derived from plants (Modulevsky et al., 2014, 2016; Contessi Negrini et al., 2020) and bacterial nanocellulose (BNC), to synthetic modified polymers (Nosar et al., 2016). These allow the diverse and versatile range of cellulose mechanical, physical, and structural properties such as morphologies, as a stand-alone or as a composite reinforcement material (Hickey and Pelling, 2019). However, in order to fully fabricate cellulose-based biomimetic tissues that require specific cell-matrix interactions, further investigations of cellulose structural properties that could mimic native tissues are studied. For instance, cellulose derived from apple hypanthium was studied for adipose tissue engineering, carrot for bone tissue engineering, celery for tendons (Contessi Negrini et al., 2020) and BNC for burns and chronic wounds treatments or in vivo implantations (Osorio et al., 2019).

Alternatively, cellulose derived from macroalgae has not been fabricated as a standalone scaffold for tissue engineering. Macroalgae, known as seaweed, have a biostable structural and biochemical advantages compared to bacterial and terrestrial plants cellulose, including high degree of cellulose crystallinity that results in higher inertness and makes seaweed cellulose less susceptible to chemical and thermal treatments. Similar to plants, green macroalgae's matrix consists of a highly robust skeleton structure that can be utilized for cell growth. Their chemical composition is rich with insoluble polysaccharides, that provides for the preservation of structural and mechanical rigidity, crystallinity, and tensile strength. Thus, their structural and biochemical variations could potentially be considered for biomedical applications that do not require biodegradability, while maintaining intact shape and form. However, unlike bacterial based-cellulose, that require strong bases treatment for the removal of microbial cells, and unlike terrestrial plants that require vertical growth, seaweed lignin-free cell-wall makes macroalgae decellularization easier and cheaper to produce. Furthermore, it grants its matrix structurally flexible yet resilient tissue, which could potentially be explored for its ECM-cell interactions and long-term sustainability.

Macroalgae have high growth rates, they are abundant and could be harvested easily all year with no need for fertilizers, which makes its mass production more affordable and macroalgae a reliable low-cost resource. Additionally, macroalgae show environmental advantages. They do not compete with food supply, land for agriculture and forestry, or freshwater supply. They help to mitigate global warming and climate change by utilizing doses of CO₂. Common macroalgae derivatives are renewable and sustainable resources for food, fuel, and chemicals applications. Furthermore, among seaweeds, red and brown algae species are largely used for their carrageenan, alginates and agaroses in tissue engineering, wound healing, and drug delivery (Kumar et al., 2019), and play a major role in biological and biomedical products. Green macroalgae derived sulfated polysaccharides (SPs), such as ulvans, too, have been proposed for tissue engineering (Madub et al., 2021). However, marine natural source of cellulose from green macroalgae have been overlooked for biomedical applications (Hickey and Pelling, 2019).

Cellulose-based ECM is a relatively new field of research, more so macroalgae-based cellulose (Wahlström et al., 2020), and little is known about its compatibility as an alternative matrix for in vitro cell culture or in vivo implantation.

Trivedi et al. (2016) discloses an integrated process that can be applied to biomass of the green seaweed Ulva fasciata, to allow the sequential recovery/extraction of economically important fractions, including cellulose which is obtained by a process other than decellularization.

Modulevsky et al. (2016) describes a study wherein the native hypanthium tissue of apples was utilized in a decellularization process to create implantable cellulose scaffolds, similar to methods used for mammalian tissues. Macroscopic cell-free cellulose biomaterials were produced and subcutaneously implanted in mouse model for a period of up to eight weeks. The work demonstrates that 3D biocompatible cellulose scaffolds may be easily produced, and that said scaffolds may become vascularized and integrated into surrounding healthy tissues.

U.S. Pat. No. 11,167,062 discloses a scaffold biomaterial comprising a decellularized plant tissue from which cellular materials and nucleic acids of the tissue are removed, wherein said scaffold has a porous structure. According to this publication, the plant referred to includes green algae (e.g., Ulva) as well as brown- and red algae. The patent discloses methods for preparing such a scaffold biomaterial, as well as uses thereof as an implantable scaffold for supporting animal cell growth, promoting tissue regeneration, promoting angiogenesis, or a tissue replacement procedure, as well as in cosmetic surgeries.

### SUMMARY OF INVENTION

In one aspect, disclosed herein is a cellulose-based scaffold comprising a decellularized macroalgae tissue from which cellular materials and nucleic acids of the tissue are removed, wherein said scaffold has a fibrous structure. In certain embodiments, the macroalgae from which said decellularized macroalgae tissue has been obtained is a green macroalgae such as a Cladophora sp. or an Ulva sp. Particular such scaffolds are free of cellular organelles and nuclei content, neither functionalized nor crosslinked, and/or free of non-biocompatible components. The cellulose-based scaffold disclosed may further comprise living animal cells, e.g., mammalian cells, adhered to said fibrous structure.

In another aspect, disclosed herein is an implant comprising a cellulose-based scaffold as defined above. In certain embodiments, the cellulose-based scaffold composing said implant is seeded with living animal cells, i.e., said implant comprises a cellulose-based scaffold to which living animal cells are adhered.

In a further aspect, disclosed herein is a decellularization process for the preparation of a cellulose-based scaffold comprising a decellularized macroalgae tissue from which cellular materials and nucleic acids of the tissue are removed, said process comprising the steps of:
(i) soaking a whole fresh macroalgae, e.g., a green macroalgae such as a Cladophora sp. or an Ulva sp., in an organic solvent such as acetone, repeatedly for several times, to thereby remove pigments, lipids, and proteins;
(ii) washing the macroalgae biomass obtained in step (i) with water, and then bleaching with a sodium chlorite (NaClO₂)-containing buffer, to thereby remove polysaccharides other than cellulose such as hemicellulose;
(iii) washing the macroalgae biomass obtained in step (ii) with water to reach a neutral pH, and then performing an alkali treatment, to thereby break down and remove lignin;
(iv) washing the macroalgae biomass obtained in step (iii) with water to reach a neutral pH, and then performing an inorganic acid treatment, to thereby remove excessive polysaccharides such as starch that might remain close to the cell wall, and optionally further allowing the biomass to rest at room temperature;
(v) washing the macroalgae biomass obtained in step (iv) in water to reach a neutral pH, to thereby obtain a clear cellulose biomass; and
(vi) optionally filtering and/or drying the cellulose biomass obtained in step (v) to thereby obtain said cellulose-based scaffold.

The process of the present invention may further comprise the step of recellularization of the cellulose-based scaffold obtained by implementing living animal cells, e.g., mammalian cells, on said cellulose-based scaffold.

In yet another aspect, disclosed herein is an implant comprising a cellulose-based scaffold obtained by the decellularization process defined above. In certain embodiments, said decellularization process comprises the step of recellularization of the cellulose-based scaffold obtained with living animal cells, and the cellulose-based scaffold composing said implant is thus seeded with said cells.

### BRIEF DESCRIPTION OF DRAWINGS

The patent or application file contains at least one drawing executed in color. Copies of this patent or patent application publication with color drawing(s) will be provided by the Office upon request and payment of the necessary fee.

FIGS. 1A-1F show thallus morphology macro view of Ulva sp. and Cladophora sp. (1A and 1D, respectively); light microscopy observation (40×) of middle region revealing Ulva sp. micro-porous structure and Cladophora sp. branching fibrous filamentous structure (1B and 1E, respectively); and hematoxylin and eosin (H&E) staining of cross-sections revealing tissue fragments of Ulva sp. by-layer porous structure and of Cladophora sp. fibers (1C and 1F, respectively). Scale bars: (1A) 2.5 cm; (1B, 1E) 20 μm, (1C, 1F) 100 μm, and (1D) 0.25 cm.

FIG. 2 schematically illustrates the decellularization treatment described in Materials and Methods. As shown, fresh macroalgae thallus samples were soaked in acetate buffer to remove pigments and proteins (step 1); soaked in bleach bath to remove polysaccharides of simpler structure than cellulose (step 2); subjected to an alkali treatment with sodium hydroxide, to remove all excessive lipids and hemicellulose within the cell wall (step 3), and then to acid treatment with hydrochloric acid, to remove all excessive polysaccharides such as starch, that might remain close to the cell wall (step 4). Following step 4, the samples were rinsed in distilled water until reaching a neutral PH and obtaining a clear clean cellulose biomass, and were then filtered and dried (step 5) making them ready to be used acellular scaffolds.

FIGS. 3A-3I show different microstructure scaffolds obtained from Ulva sp. and Cladophora sp. following the decellularization treatment described in Study 1, Materials and Methods, wherein the drying step is carried out at room temperature on a flat surface for a period of 4-7 days (3A, Ulva sp.; 3B, Cladophora sp.); at 40° C. in an oven for 24-48 h (as shown in FIG. 2); or using a lyophilizer (−80° C.) at various biomass to distilled water ratios (3E). Fiber-like structure, paper-like structure, ˜50 mm thick cotton-like sponge, −200 μm thickness silk-like thin film, and cardboard-like structure are shown in 3C, 3D, 3E, 3F, and 3H, respectively. 3G shows decellularized non-dried raw material. 3I shows exact shapes (1 cm² squares, 2 mm diameter circles) cut by laser for easy-to-use scaffolds, as well as negative space of circles leftovers.

FIGS. 4A-4L show structural surface area SEM imaging of Ulva sp. acellular scaffold, indicating highly organized porous architecture with average pore size width of 20.2±4 μm (n=50 analyzed regions) and cell wall thickness of from 0.5 to 2.0 μm (n=10 analyzed regions) (4A-4C); and of Cladophora sp. acellular scaffold, indicating highly fibrous architecture with fiber diameter from 5 μm to above 80 μm (n=55 analyzed regions), covered with microfibrils ranging in width from 55 to 400 nm (n=50 analyzed regions) (4G-4I). H&E staining of cross-sections of decellularized scaffolds of Ulva sp. and Cladophora sp. reveals eosin stain of the matrix but no hematoxylin (cell nucleus) (4D and 4J, respectively). Corresponding fluorescent microscopy images of the seaweed cell wall stained with Calcofluor White reveals middle region overview structural properties and confirms cellulose as the prime structural component of the seaweed scaffold of Ulva sp. and Cladophora sp. (4E and 4K, respectively). Both seaweed scaffolds were confirmed acellular, i.e., empty of cell organelles, indicating that the decellularization method was effective, and that the seaweed cellulose structural shape remained intact post decellularization treatment. 3F and 3L show macro view of the decellularized seaweed (Ulva sp. and Cladophora sp. used as scaffolds for cell growth, respectively. *Scale bars: (4A, 4G) 50 μm, (4B, 4H) 20 μm, (4C, 4I) 5 (4D, 4J) 100 μm, (4E, 4K) 10 μm, (4F, 4L) 0.25 cm.

FIG. 5 shows DNA quantification gel electrophoresis analysis of fresh and decellularized seaweed samples of Ulva sp. and Cladophora sp., using a plant genomic DNA purification method (n=3 for each sample). DNA concentrations for Ulva sp. and Cladophora sp. were measured using a NanoDrop spectrophotometer, as described in Study 1, Materials and Methods; and gel electrophoresis was then used to confirm the results. Oval dashed circles indicate the high DNA concentrations of the fresh seaweed samples. Fresh Ulva (FU), fresh Cladophora (FC), decellularized Ulva (DU) and Cladophora (DC).

FIGS. 6A-6F show SEM imaging of sterilized cellulose scaffolds recellularized with fibroblast, after 4 weeks of seeding, indicating cell growth and cell attachments onto the Ulva sp. porous matrix, with average cell size of 34.2±8.4 μm (n=40 analyzed regions) (6A-6C), and along the Cladophora sp. fibrous matrix, with average cell size of 20.1±4 μm (n=70 analyzed regions) (6D-6F). Observations show elongated filament profusions tracing the Ulva sp. porous cell-wall matrix (6C) and along the Cladophora sp. fibers (6D), as well as connected to neighboring cells on both scaffolds' surface areas, and confirm cell-to-matrix and cell-to-cell interactions in both cases. *Scale bars: (6A, 6D) 200 μm, (6B, 6F) 20 μm, (6C) 10 μm, (6E) 50 μm.

FIGS. 7A-7F show fluorescence confocal microscopy imaging of live fibroblast (20×10³ cells/μl) labeled with actin-GFP (green), overlaying the macroalgae cellulose scaffolds, detected in reflection mode. 3D Z-stack and orthogonal views (40×) reveal cell growth and attachments onto the Ulva sp. porous matrix (Day 41) (7A-7B) and Cladophora sp. fibrous matrix (Day 42) (7C-7D, respectively). Dash lines indicate the location of the orthogonal cut. Additional time-lapse imaging (20x) reveals cell growth and spreading on the cellulose scaffolds of Ulva sp. (Day 32) and Cladophora sp. (Day 40) (7E and 7F, respectively). Extended slender cell protrusions observed on both scaffolds, indicate that the cells remain alive and function during the entire experiment as they formed connectivity with neighboring cells and the scaffolds' surface area. *Scale bars: (7A, 7B, 7E) 50 μm, (7C, 7D) 30 μm, (7F) 80 μm.

FIG. 8 shows the relative change of alamarBlue (AB) fluorescence signal, directly reflecting the metabolic activity of the cell culture, after incubating fibroblasts with with 30% and 100% media extracts from Ulva sp. (U 100% and U 30%) and Cladophora sp. (Cl 100% and Cl 30%) cellulose scaffolds, at incubation time points t=24, 48 and 72 hours. Control groups include negative control of cell culture incubated with regular media (Ctrl cells) and positive cytotoxic 70% methanol treatment (70% M). The dashed line at 70% viability, distinguish between viable and toxic constructs. Values are expressed as mean±SD, n=5, *p<0.05 (obtained by Student t-test).

FIGS. 9A-9B show plots representing growth of fibroblast seeded with seaweed cellulose scaffolds derived from Ulva sp. (9A) and Cladophora sp. (9B), at initial cell concentrations of 5×10³, 10×10³, 20×10³ and 40×10³ cells/μl (upper left-, upper right-, lower left-, and lower right-panel, respectively), over a period of 40 days, relative to the alamarBlue percentage reduction. Control groups include Ulva sp. and Cladophora sp. scaffolds without cells (U Ctrl, Cl Ctrl), Blank media, and 10% AB media solution. Values are expressed as mean±SD, n=3. Dashed lines represent the prediction model.

FIGS. 10A-10D show modeled cell proliferation rates as a function of initial cell seeding concentration for the Ulva sp., which reached rapid increase following by cell saturation at high cell concentrations (10A), and for Cladophora sp., with a linear increase correlated to cell proliferation rate and initial cell seeding concentrations (10B). Further shown are scheme of cell migration and alignment in correlation to SC structures, wherein the Ulva sp. matrix facilitates migration opportunities in all directions, resulting in rapid cell growth, as cells ‘cover’ the scaffold's microporous surface area, following proliferation rate decrease, due to early cell saturation (10C); and the Cladophora sp. structure facilitates migration opportunities along the fiber elongated axis, guided by microfibrils that overlay the fiber's surface, resulting in linear increase of proliferation rates (10D).

FIG. 11 shows scaffold and tissue resection and collection as described in Study 2. The dorsal skin surgical sites (n=2, left and right) (left panel) were dissected (1-2 cm² sq) (middle panel), and collected for further histological analysis (right panel).

FIGS. 12A-12C show tissue cross sections of SC Ulva sp. porous implant (12A), Cladophora sp. fibrous implant (12B) and sections of control group with no implant (12C), stained with H&E, for the evaluation of biocompatibility, cell infiltration and foreign body reaction (FBR), including cell type and cell response, at week 1, 4 and 8. SC implants (12A, 12B) top row reveal global view, and bottom row reveal representative view of implant center ROI. *Scale bars: top row=1 mm, bottom row=100 μm.

FIGS. 13A-13C show tissue cross sections of SC Ulva sp. porous implant (13A), Cladophora sp. fibrous implant (13B) and sections of control group with no implant (13C), stained with Masson's Trichrome, for the evaluation of extracellular matrix deposition, including fibroplasia, fibrosis, and neovascularization at week 1, 4 and 8. Top row reveal global view with fibrosis capsule of both SC implants, and bottom row reveal representative view of implant center ROI, with angiogenesis. *Scale bars: top row=1 mm, bottom row=100 μm.

FIGS. 14A-14C show tissue cross sections of SC Ulva sp. porous implant (14A), Cladophora sp. fibrous implant (14B) and sections of control group with no implant (14C), stained with Anti-CD31/PECAM-1 immunohistochemistry staining, for the evaluation of vascularization and angiogenesis, at week 8. Top rows reveal global view, middle (×20) and bottom (×40) row, reveal representative view of center ROI. Imaging of both SC implant sites (14A, 14B) show the presence of endothelial cells, confirming angiogenesis process with multiple blood vessels within the implant sites. *Scale bars: top row=1 mm, middle row=100 μm and bottom row=50 μm.

### DETAILED DESCRIPTION

In one aspect, the present invention provides a cellulose-based scaffold comprising a decellularized macroalgae tissue, also referred to herein as decellularized seaweed tissue, from which cellular materials and nucleic acids of the tissue are removed, wherein said scaffold has a fibrous structure, more particularly a three-dimensional fibrous structure.

The cellulose-based scaffold disclosed herein is thus a cellulose-containing fibrous structure, obtained upon removal of cellular materials and nucleic acids (decellularization) from any cellulose-containing macroalgae, i.e., macroalgae that is rich in cellulose in its cell walls. Examples of such macroalgae include green macroalgae, red macroalgae, and brown macroalgae.

In certain embodiments, the decellularized seaweed tissue composing the cellulose-based scaffold disclosed herein is obtained after decellularization of a green seaweed tissue, e.g., a tissue of a Cladophora species (Cladophora sp.) or a mixture thereof (Cladophora spp.). Examples of Cladophora spp. include, without being limited to, Cladophora albida, Cladophora aokii, Cladophora brasiliana, Cladophora catenata, Cladophora coelothrix, Cladophora columbiana, Cladophora crispata, Cladophora dalmatica, Cladophora fracta, Cladophora glomerata, Cladophora graminea, Cladophora montagneana, Cladophora ordinata, Cladophora prolifera, Cladophora rivularis, Cladophora rupestris, Cladophora scopaeformis, Cladophora sericea, Cladophora socialis, and Cladophora vagabunda.

In other embodiments, the decellularized seaweed tissue composing the cellulose-based scaffold disclosed herein is obtained after decellularization of a red seaweed tissue, e.g., a tissue of a Bangia species (Bangia sp.) or a mixture thereof (Bangia spp.). Examples of Bangia spp. include, without limiting, Bangia aeruginosa, Bangia amethystina, Bangia anisogona, Bangia annulina, Bangia atropurpurea, Bangia atrovirens, Bangia biseriata, Bangia breviarticulata, Bangia callicoma, Bangia carnea, Bangia coccineopurpurea, Bangia condensata, Bangia confervoides, Bangia crispula, Bangia discoidea, Bangia dura, Bangia enteromorphoides, Bangia fergusonii, Bangia ferruginea, Bangia flocculosa, Bangia foetida, Bangia foetida, Bangia foliacea, Bangia fulvescens, Bangia fuscopurpurea, Bangia gloiopeltidicola, Bangia grateloupicola, Bangia halymeniae, Bangia harveyi, Bangia homotrichoides, Bangia intricata, Bangia intricata, Bangia kerkensis, Bangia lacustris, Bangia lanuginosa, Bangia latissima, Bangia malacensis, Bangia maxima, Bangia punctulata, Bangia purpurea, Bangia quadripunctata, Bangia radicula, Bangia sericea, Bangia simplex, Bangia tanakae, Bangia tavarisii, Bangia tenuis, Bangia thaerasiae, Bangia trichodes, Bangia vermicularis, Bangia viridis, and Bangia yamadae.

In certain embodiments, the decellularized seaweed tissue composing the cellulose-based scaffold disclosed herein is obtained after decellularization of a brown seaweed tissue.

In certain embodiments, the macroalgae tissue composing the cellulose-based scaffold disclosed herein is obtained after decellularization of a tissue of a Cladophora sp., e.g., one of the Cladophora species listed above or Cladophora spp., e.g., a mixture of two or more Cladophora species of those listed above.

The cellulose-based scaffold disclosed herein has a three-dimensional fibrous structure. In certain embodiments, said fibrous structure, according to any one of the embodiments above, is highly packed, i.e., consists of highly dense fibers, and has the form of a threadlike (web-like) filamentous matrix.

In particular such embodiments, said fibrous structure comprises heterogeneous fibers comprising fibers having a width of from about 0.5 μm to about 800 μm, e.g., from about 1 μm to about 700 μm, from about 2 μm to about 600 μm, from about 3 μm to about 500 μm, from about 4 μm to about 400 μm, or from about 5 μm to about 300, 200, or 100 μm, but preferably from about 5 μm to about 80 μm; microfibrils having a width of from about 0.55 nm to about 4 μm, e.g., from about 1 nm to about 2 μm, from about 2 nm to about 1 μm, from about 3 nm to about 800 nm, or from about 4 nm to about 600 nm, but preferably from about 55 nm to about 400 nm; and/or a combination thereof. In more particular such embodiments, said fibrous structure comprises a combination of both fibers having a width of from about 0.5 μm to about 800 μm, preferably from about 5 μm to about 80 μm; and microfibrils having a width of from about 0.55 nm to about 4 μm, preferably from about 55 nm to about 400 nm.

In certain embodiments, the cellulose-based scaffold disclosed herein, according to any one of the embodiments above, is free or essentially free of cellular organelles and nuclei content.

The phrase “essentially free of cellular organelles and nuclei content” as used herein with respect to the cellulose-based scaffold disclosed means that said scaffold may comprise residual cellular components such as DNA, mitochondria, and membrane-associated molecules including phospholipids, rather than being completely free of such material, considering that decellularization techniques usually cannot completely remove such material. The cellular components optionally comprised within the scaffold, i.e., left following the decellularization process utilized, can be measured quantitatively.

According to the literature, residual cellular material within a decellularized extracellular matrix (ECM) may contribute to cytocompatibility problems in vitro and adverse host responses in vivo upon reintroduction of cells. The threshold concentration of residual cellular material within the decellularized ECM sufficient to elicit a negative remodeling response may vary depending upon the ECM source, the type of tissue into which the ECM is implanted, and the host immune function. Based on Crapo et al., 2011, the minimal criteria for avoiding adverse cell and host responses upon in vitro use and implantation of a decellularized ECM is: (i)<50 ng DNA per mg ECM dry weight, as confirmed by, e.g., DNA quantification analysis; (ii)<200 bp DNA fragment length; and/or (iii) lack of visible nuclear material in tissue sections of the decellularized ECM, as confirmed by, e.g., staining with 4′,6-diamidino-2-phenylindole (DAPI) or histology analysis using Hematoxylin and Eosin (H&E).

In certain embodiments, the cellulose-based scaffold disclosed herein, according to any one of the embodiments above, is neither functionalized nor crosslinked. The term “functionalized” as used herein with respect to a cellulose-based scaffold means that said scaffold is chemically, i.e., covalently, modified, e.g., by acylation and/or alkylation at some of the free hydroxyl groups thereof. Such a modified cellulose-based scaffold may also be referred to as “a coated cellulose-based scaffold”. A covalent modification of a cellulose-based scaffold with a functional chemical group, i.e., a group capable of undergoing a chemical reaction, may enable linking to said scaffold, through said functional group, an active agent such as a drug or a growth factor. The term “crosslinked” as used herein with respect to a cellulose-based scaffold means that at least two hydroxyl groups of either a cellulose molecule or adjacent cellulose molecules of said cellulose-based scaffold are linked, following a reaction of a non-crosslinked cellulose-based scaffold with a cross-linking agent, i.e., a multi-functional agent having at least two functional groups, e.g., a bifunctional agent, capable of reacting with, and consequently linking, two hydroxyl groups. Using a cross-linking agent having more than two functional groups, e.g., a tri-functional agent, may further enable linking to said scaffold, through a functional group of said cross-linking agent, an active agent such as a drug or a growth factor. A cellulose-based scaffold may be crosslinked so as to, e.g., enhance the dimensional stability of said scaffold. A non-functionalized, non-crosslinked cellulose-based scaffold thus denotes such a scaffold in which all the hydroxyl groups are free.

In certain embodiments, the cellulose-based scaffold disclosed herein, according to any one of the embodiments above, is free of non-biocompatible components, i.e., a material capable of producing a toxic or immunological response when exposed to the body or a bodily fluid (e g, amniotic fluid, aqueous humour, vitreous humour, bile, blood serum, breast milk, cerebrospinal fluid, pleural fluid, cerumen, endolymph, perilymph, female ejaculate, gastric juice, mucus, peritoneal fluid, saliva, sebum, semen, sweat, tears, vaginal secretion, urine, and pus) of a mammalian, e.g., a human.

In certain embodiments, the cellulose-based scaffold of the present invention, according to any one of the embodiments above, is seeded with living animal cells, i.e., further comprises living animal cells adhered to said fibrous structure. In particular embodiments, said living animal cells are mammalian cells, e.g., human cells. Examples of mammalian cells that might be adhered to the cellulose-based scaffold disclosed herein include, without limiting, fibroblasts, myoblasts, endothelial cells, vascular cells, umbilical vein endothelial cells (UVEC), adipose cells such as adipose mesenchymal stem cells, and hematopoiesis stem cells, e.g., human fibroblasts, human myoblasts, human endothelial cells, human vascular cells, human umbilical vein endothelial cells (HUVEC), human adipose cells such as human adipose mesenchymal stem cells, and human hematopoiesis stem cells.

In particular embodiments, the living animal cells adhered to the cellulase-based scaffold have an average diameter of from about 10 μm to about 100 μm, e.g., from about 12 μm to 90 μm, from about 14 μm to 80 μm, from about 16 μm to 70 μm, from about 18 μm to 60 μm, or from about 20 μm to 50 μm. In particular such embodiments, the cellulose-based scaffold disclosed herein is seeded with fibroblasts, e.g., human fibroblasts, having an average diameter of about 20 μm.

The cellulose-based scaffolds of the present invention, and particularly when not seeded with living animal cells, may be used in non-biomedical applications, e.g., for design products, building products, and/or food products, as an alternative material to paper-based, plastic-based, or foam-based materials, or as a three-dimensional (3D) printing material. In addition, such cellulose-based scaffolds may be used in biomedical applications, e.g., for encapsulation, wound dressing, or as implants for use in vivo.

In another aspect, the present invention thus provides an implant comprising a cellulose-based scaffold as disclosed herein, i.e., a cellulose-based scaffold comprising a decellularized macroalgae tissue and having a fibrous structure, according to any one of the embodiments above, e.g., such a cellulose-based scaffold that is neither functionalized nor crosslinked. In certain embodiments, the implant disclosed herein comprises a cellulose-based scaffold seeded with living animal cells, i.e., a cellulose-based scaffold to which living animal cells such as mammalian (e.g., human) cells, are adhered.

The terms “implant”, “cellulose-based biomimetic tissue”, “cellulose-based extracellular matrix (ECM)”, and “cellulose-based biocompatible biomaterial” are used herein interchangeably and refer to a construct comprising a cellulose-based scaffold as disclosed herein, which may be used for, e.g., supporting living animal cells growth, promoting tissue regeneration, promoting angiogenesis, and tissue replacement, or as a structural implant for, e.g., a cosmetic surgery.

In certain embodiments, the implant disclosed herein according to any one of the embodiments above may be used as, e.g., a structural implant for tissue repair or regeneration following spinal cord injury; a structural implant for tissue replacement surgery and/or for tissue regeneration following a surgery; a structural implant for skin graft and/or a skin regeneration surgery; a structural implant for regeneration of blood vasculature in a target tissue or region; a bone replacement, bone filling, or bone graft material, and/or for promoting bone regeneration; a tissue replacement for skin, bone, spinal cord, heart, muscle, nerve, blood vessel, or other damaged or malformed tissue; a vitreous humour replacement (e.g., in a hydrogel form); an artificial bursae; and a structural implant for a cosmetic surgery.

In other embodiments, the implant disclosed herein according to any one of the embodiments above may be used in cultured meat (also referred to as “cultivated meat” or “cell-based meat”) production.

In a further aspect, the present invention relates to a decellularization process for the preparation of a cellulose-based scaffold comprising a decellularized macroalgae (seaweed) tissue from which cellular materials and nucleic acids of the tissue are removed, e.g., a cellulose-based scaffold having a fibrous structure as disclosed herein, said process comprising the steps of:
(i) soaking a whole fresh macroalgae (fresh macroalgae thallus) in an organic solvent, repeatedly for several times (e.g., up to 4 or 5 times), to thereby remove pigments, lipids, and proteins;
(ii) washing the macroalgae biomass obtained in step (i) with water (e.g., doubly distilled water), and then bleaching with a sodium chlorite (NaClO₂)-containing buffer, to thereby remove polysaccharides other than cellulose such as hemicellulose;
(iii) washing the macroalgae biomass obtained in step (ii) with water (e.g., doubly distilled water) to reach a neutral pH, and then performing an alkali treatment, to thereby break down and remove lignin;
(iv) washing the macroalgae biomass obtained in step (iii) with water (e.g., doubly distilled water) to reach a neutral pH, and then performing an inorganic acid treatment, to thereby remove excessive polysaccharides such as starch that might remain close to the cell wall, and optionally further allowing the biomass to rest, e.g., overnight, at room temperature;
(v) washing the macroalgae biomass obtained in step (iv) in water (e.g., doubly distilled water) to reach a neutral pH, to thereby obtain a clear cellulose biomass; and
(vi) optionally filtering and/or drying the cellulose biomass obtained in step (v) to thereby obtain said cellulose-based scaffold.

The term “organic solvent” as used herein refers to a polar solvent that is miscible in water, e.g., acetone and alcohols such as methanol, ethanol, isopropanol, and glycerol.

In certain embodiments, step (i) of the process disclosed herein is performed at a temperature ranging from room temperature to the boiling temperature of the organic solvent in which said fresh macroalgae is soaked. In particular embodiments, the organic solvent utilized in step (i) is acetone, and said step is performed at room temperature for at least few (e.g., 2, 3, 4, or 5) days, or at about 60° C. for about 60 minutes.

As found by the present inventors, soaking a fresh macroalgae in acetone in step (i) of the process enabled dehydrating the macroalgae before proceeding to the next steps, as well as removing pigments, lipids, and soluble components from the macroalgae. Moreover, the fact that the decellularization process disclosed herein is carried out on a fresh macroalgae, rather than dry (either grinded/crushed or not) material enables obtaining a whole acellular cellulose-based scaffold, i.e., retaining the native structure of the tissue of the macroalgae.

In certain embodiments, the bleaching in step (ii) of the process disclosed herein, according to any one of the embodiments above, is performed with an acetate buffer, at a temperature of about 60° C. In particular embodiments, said acetate buffer is based on sodium acetate (NaAc) and acetic acid (AcOH), and comprises about 20% w/v sodium chlorite.

In certain embodiments, the alkali treatment in step (iii) of the process disclosed herein, according to any one of the embodiments above, is performed with, e.g., about 0.1-1M, about 0.2-0.8M, about 0.4-0.6M, or about 0.5M, sodium hydroxide, at a temperature of about 40° C. to about 80° C., e.g., at about 50, 60 or 70° C. In particular embodiments, said alkali treatment is performed with 0.5M sodium hydroxide, at 60° C., for at least about 8 hours.

The inorganic acid treatment in step (iv) of the process disclosed herein may be carried out with any suitable inorganic acid, i.e., with any inorganic acid capable of removing excessive polysaccharides such as starch, from the macroalgae biomass obtained in step (iii). In certain embodiments, said inorganic acid treatment, according to any one of the embodiments above, is performed with hydrochloric acid, e.g., at a concentration of about 1-10% v/v, about 2-8% v/v, 4-6% v/v, or about 5% v/v, and at a temperature of about 100° C., preferably for a few minutes, e.g., up to about 5, 10, 15, or 20 minutes. In particular embodiments, said acid treatment is carried out with 5% v/v hydrochloric acid at about 100° C., for about 10 minutes or until boiling starts. The macroalgae biomass obtained following the inorganic acid treatment may optionally be left to rest in said inorganic acid for several hours (e.g., for about 3, 4, 5, 6, 7, or 8 hours) or overnight at room temperature.

In certain embodiments, the macroalgae biomass obtained in one or more of steps (i)-(v) of the process disclosed herein, according to any one of the embodiments above, is stored under refrigerated conditions, i.e., at 2-8° C., e.g., at about 4° C., before being subjected to the next step.

In certain embodiments, the macroalgae biomass obtained in step (v) of the process disclosed herein, according to any one of the embodiments above, is filtered, e.g., with a nylon filter; and/or dried, e.g., at about 40° C. in an oven for at least 24 hours, at room temperature for up to several days, or by freeze-drying.

In a particular such aspect thus disclosed herein a process for the preparation of a cellulose-based scaffold comprising a decellularized macroalgae tissue from which cellular materials and nucleic acids of the tissue are removed, said process comprising the steps of:
(i) soaking a whole fresh macroalgae (fresh macroalgae thallus) in acetone, repeatedly for several times (e.g., up to 4 or 5 times), at either room temperature for at least few (e.g., 2, 3, 4, or 5) days or at about 60° C. for about 60 minutes, to thereby remove pigments, lipids, and proteins;
(ii) washing the macroalgae biomass obtained in step (i) with water (e.g., doubly distilled water), and then bleaching with a sodium chlorite (NaClO₂)-containing acetate buffer, at a temperature of about 60° C., to thereby remove polysaccharides other than cellulose such as hemicellulose;
(iii) washing the macroalgae biomass obtained in step (ii) with water (e.g., doubly distilled water) to reach a neutral pH, and then performing an alkali treatment with sodium hydroxide (0.5M), at a temperature of about 60° C., to thereby break down and remove lignin;
(iv) washing the macroalgae biomass obtained in step (iii) with water (e.g., doubly distilled water) to reach a neutral pH, and then treating with hydrochloric acid (e.g., 5% v/v), at a temperature of about 100° C., preferably for up to 5, 10, 15, or 20 minutes, to thereby remove excessive polysaccharides such as starch that might remain close to the cell wall, and optionally further allowing the biomass to rest for several hours (e.g., for about 3, 4, 5, 6, 7, or 8 hours) or overnight at room temperature;
(v) washing the macroalgae biomass obtained in step (iv) in water (e.g., doubly distilled water) to reach a neutral pH, to thereby obtain a clear cellulose biomass; and
(vi) optionally filtering the cellulose biomass obtained in step (v), e.g., with a nylon filter, and/or drying said biomass obtained, e.g., by freeze drying, at room temperature, or at a temperature above room temperature, to thereby obtain said cellulose-based scaffold.

In certain embodiments, the macroalgae treated by the process of the present invention, according to any one of the embodiments above, is a green macroalgae, e.g., a Cladophora species or a mixture thereof, or an Ulva species (Ulva sp.) or a mixture thereof (Ulva spp.). Examples of Cladophora spp. are listed above, and examples of Ulva spp. include, without being limited to, Ulva acanthophora, Ulva anandii, Ulva arasakii, Ulva atroviridis, Ulva australis, Ulva beytensis, Ulva bifrons, Ulva brevistipita, Ulva burmanica, Ulva californica, Ulva chaetomorphoides, Ulva clathrata, Ulva compressa, Ulva conglobata, Ulva cornuta, Ulva covelongensis, Ulva crassa, Ulva crassimembrana, Ulva curvata, Ulva denticulata, Ulva diaphana, Ulva elegans, Ulva enteromorpha, Ulva erecta, Ulva expansa, Ulva fasciata, Ulva flexuosa, Ulva geminoidea, Ulva gigantea, Ulva grandis, Ulva hookeriana, Ulva hopkirkii, Ulva howensis, Ulva indica, Ulva intestinalis, Ulva intestinaloides, Ulva javanica, Ulva kylinii, Ulva lactuca, Ulva laetevirens, Ulva laingii, Ulva linearis, Ulva linza, Ulva lippii, Ulva litoralis, Ulva littorea, Ulva lobata, Ulva marginata, Ulva micrococca, Ulva mutabilis, Ulva neapolitana, Ulva nematoidea, Ulva ohnoi, Ulva olivascens, Ulva pacifica, Ulva papenfussii, Ulva parva, Ulva paschima, Ulva patengensis, Ulva percursa, Ulva pertusa, Ulva phyllosa, Ulva polyclada, Ulva popenguinensis, Ulva porrifolia, Ulva profunda, Ulva prolifera, Ulva pseudocurvata, Ulva pseudolinza, Ulva pulchra, Ulva quilonensis, Ulva radiata, Ulva ralfsii, Ulva ranunculata, Ulva reticulata, Ulva rhacodes, Ulva rigida, Ulva rotundata, Ulva saifullahii, Ulva serrata, Ulva simplex, Ulva sorensenii, Ulva spinulosa, Ulva stenophylla, Ulva sublittoralis, Ulva subulata, Ulva taeniata, Ulva tanneri, Ulva tenera, Ulva torta, Ulva tuberosa, Ulva uncialis, Ulva uncinata, Ulva usneoides, Ulva utricularis, Ulva utriculosa, Ulva uvoides, and Ulva ventricosa.

In certain embodiments, the macroalgae treated by the process of the invention is a green macroalgae of the genus Cladophora, or a mixture of more than one such species, e.g., one of the Cladophora species listed above or a mixture thereof, and the scaffold obtained by said process has a fibrous structure, more specifically a three-dimensional fibrous structure, e.g., having the form of a filamentous matrix. Such a fibrous structure may comprise heterogeneous fibers comprising fibers having a width of from about 0.5 μm to about 800 μm, preferably from about 5 μm to about 80 μm; or microfibrils having a width of from about 0.55 nm to about 4 μm, preferably from about 55 nm to about 400 nm; but preferably a combination thereof.

In other embodiments, the macroalgae treated by the process of the invention is a green macroalgae is of the genus Ulva, or a mixture of more than one such species, e.g., one of the Ulva species listed above or a mixture thereof, and the scaffold obtained by the process of the present invention has a porous structure, more specifically a three-dimensional porous structure. Such a porous structure may have interconnected cellulose web-like polygonal pattern; may comprise uniform pore size width in the range of about 1-50 μm; and may have cell wall thickness in the range of from about 0.1-10 μm.

In certain embodiments, the cellulose-based scaffold obtained by the process disclosed herein, according to any one of the embodiments above, is neither functionalized nor crosslinked. In other embodiments, the cellulose-based scaffold obtained by said process, according to any one of the embodiments above, is free of non-biocompatible components.

In certain embodiments, the process of the present invention, according to any one of the embodiments above, further comprises the step of recellularization of said cellulose-based scaffold by, e.g., implementing living animal cells (e.g., mammalian such as human cells) on said cellulose-based scaffold. Examples of such mammalian cells include, without limiting, fibroblasts, myoblasts, umbilical vein endothelial cells (UVEC), and adipose mesenchymal stem cells, e.g., human fibroblasts, human myoblasts, HUVEC, and human adipose mesenchymal stem cells. According to the present invention, the scaffold obtained from the specific macroalgae enables said living animal cells to reach an average cell size of from about 10 μm to about 100 μm.

The term “recellularization” as used herein refers to a process for reseeding a cellulose-based scaffold comprising a decellularized tissue from which cellular materials and nucleic acids of the tissue are removed, with cells by, e.g., static- or dynamic cell-seeding.

The term “static cell-seeding” as used herein refers to a process wherein a cell suspension is deposited on the scaffold surface and the cells are then allowed to infiltrate the scaffold. The term “dynamic cell-seeding” as used herein refers to, e.g., a rotational seeding technique carried out using hydrostatic forces, or a vacuum seeding technique based on pressure differentials, both aimed at increasing cell seeding efficiency, uniformity, and/or penetration of the scaffold.

In yet another aspect, the present invention provides an implant, i.e., a biomimetic tissue or biocompatible biomaterial, which comprises a cellulose-based scaffold obtained by the process disclosed herein, according to any one of the embodiments above. In certain embodiments, said process comprises the step of recellularization of the cellulose-based scaffold obtained, and said implant thus comprises a cellulose-based scaffold implemented with living animal cells, e.g., human cells.

Implants as disclosed herein, i.e., those disclosed per se, which comprise a cellulose-based scaffold having a fibrous structure; and those obtained by the process disclosed above, which comprise a cellulose-based scaffold having either a fibrous or porous structure, may be used either in vitro, e.g., for supporting living animal cells growth; or in vivo, when seeded with living animal cells as defined above, or at least functionalized with a growth medium aimed at stimulating cells toward endogenous tissue repair (to thereby promote, e.g., tissue regeneration and/or angiogenesis; or as a structural implant for, e.g., a cosmetic surgery).

Unless otherwise indicated, all numbers referring, e.g., to the size of fibers and/or microfibers, living animal cells, pores, all as disclosed herein, or to temperatures used in the process of the invention, used in the present specification are to be understood as being modified in all instances by the term “about”. Accordingly, unless indicated to the contrary, the numerical parameters set forth in this description and claims are approximations that may vary by up to plus or minus 10% depending upon the desired properties sought to be obtained by the invention.

The invention will now be illustrated by the following non limiting Examples.

### Examples

Study 1. SC Scaffolds Derived from Green Macroalgae for Tissue Engineering

### Materials and Methods

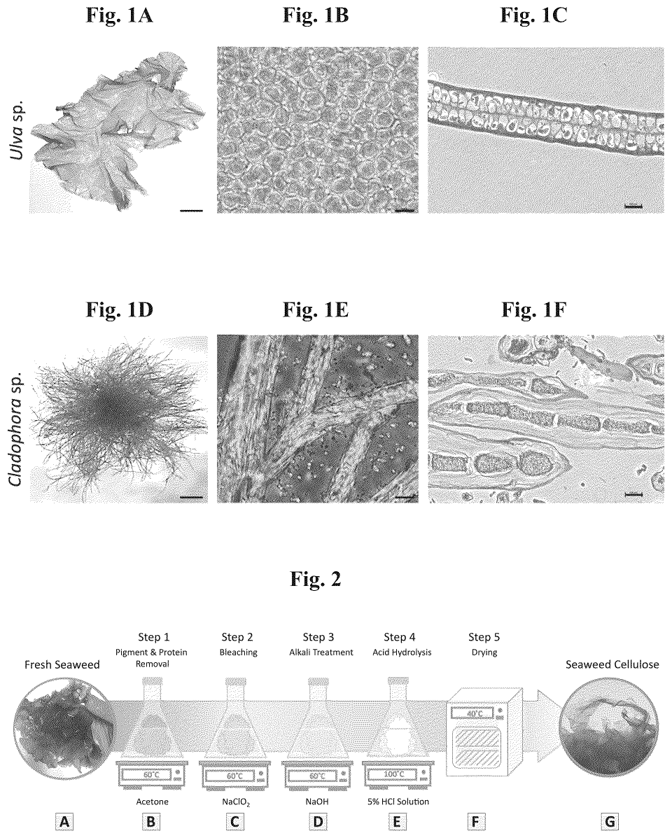

Preparation of materials. Green marine macroalgae species Ulva sp. and Cladophora sp. were used as a model for their structural composition variation: a porous and a fibrous matrix structure, respectively (FIGS. 1A-1F). These two species, which have worldwide distribution, are found in the intertidal and shallow waters of the Israeli Mediterranean seashores. Ulva sp. and Cladophora sp. are known for their fast growth rates, and are considered as potential feedstock for biorefineries. Cladophora sp. was cultivated under controlled conditions using cylindrical, sleeve-like macroalgae photo-bioreactors (MPBR, Polytiv, Israel), with sleeve dimensions of 100 cm length, 200 μm thickness, 40 cm width, and total circulation volume of 3400 L seawater (salinity 3.9%, pH 8.2). Ulva sp. was obtained from the seaweed unit of Israel Oceanographic and Limnological Research, Haifa, Israel (IOLR) and Tel Aviv University. Collected biomass from Haifa IOLR was transported to the laboratory in plastic bags filled with seawater. All samples were cleaned, sorted manually to get clean monocultures and documented for their morphology and histology.

Seaweed cellulose decellularization. A whole organ or tissue decellularization approach is a process that is used to isolate the ECM of a tissue from its inhabiting cells, leaving a “ghost” ECM scaffold of the original tissue (Crapo et al., 2011). Following an efficient decellularization treatment (Trivedi et al., 2016) and its optimization for a whole tissue culture, cellular content was extracted from the two macroalgae species Ulva sp. and Cladophora sp. (FIG. 2). Fresh algae biomass samples were obtained, cleaned and sorted by hand. 100 g wet weight Ulva sp. and Cladophora sp. seaweed samples were boiled in acetone bath (20% w/v) at 60° C. for 60 min, repeatedly 4 times, in order to remove pigments (chlorophyll) and proteins (FIG. 2, step 1). Residual biomass was boiled in acetate buffer bath, containing 1.17 g sodium chlorite (NaClO₂) (20% w/v), at 60° C. for 6-8 h, spurring bleaching and the removal of simpler structure polysaccharides (FIG. 2, step 2). The bleached seaweed residues were pH neutralized by washing with distilled water, and then alkylated in 0.5 M sodium hydroxide (NaOH) bath (20% w/v), at 60° C. for 8-10 h, to remove all excessive lipids (FIG. 2, step 3). Following the alkali treatment, the seaweed residues were pH neutralized by washing with distilled water, and then acidified in hydrochloric acid (HCl) (5% v/v), at 100° C. for 10 min (20% w/v), or until boiling started (FIG. 2, step 4). Next, samples were rested overnight at room temperature to remove all excessive polysaccharides that might remain close to the cell wall. Finally, the samples were carefully rinsed repeatedly in distilled water, until reaching a neutral pH (SevenExcellence pH Meter).

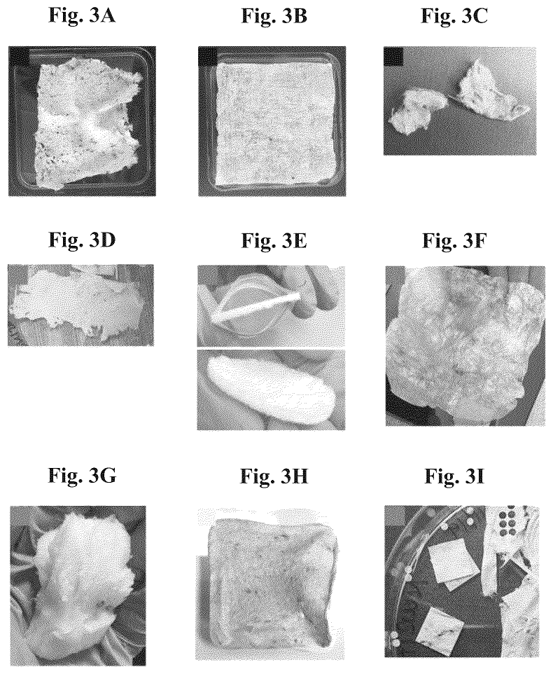

Seaweed cellulose (SC) scaffold fabrication. Obtaining a clear clean cellulose biomass, the seaweed residues were then filtered, and dried at 40° C. in an oven for 24-48 h (FIG. 2, step 5), at room temperature on a flat surface for a period of 4-7 days, or using a lyophilizer (Labconco, −80° C.) at various biomass to distilled water ratios, obtaining a final whole-tissue cellulose scaffold, having different microstructure platforms including˜200 μm thickness silk-like thin films, −50 mm thick cotton-like sponge, fiber-like structure, paper-like structure, and cardboard-like structure, ready to be used for cell growth (FIG. 3). For research replicates, exact squares and circles shapes were laser-cut for easy-to-use scaffolds. Controlling the SC fabrication, size and shapes achieved scaffold that could be tailored for various applications with a range of physio-mechanical properties, either as stand-alone agent-free or pre-cellularized bioactive carriers, or as a composite with other biomaterials.

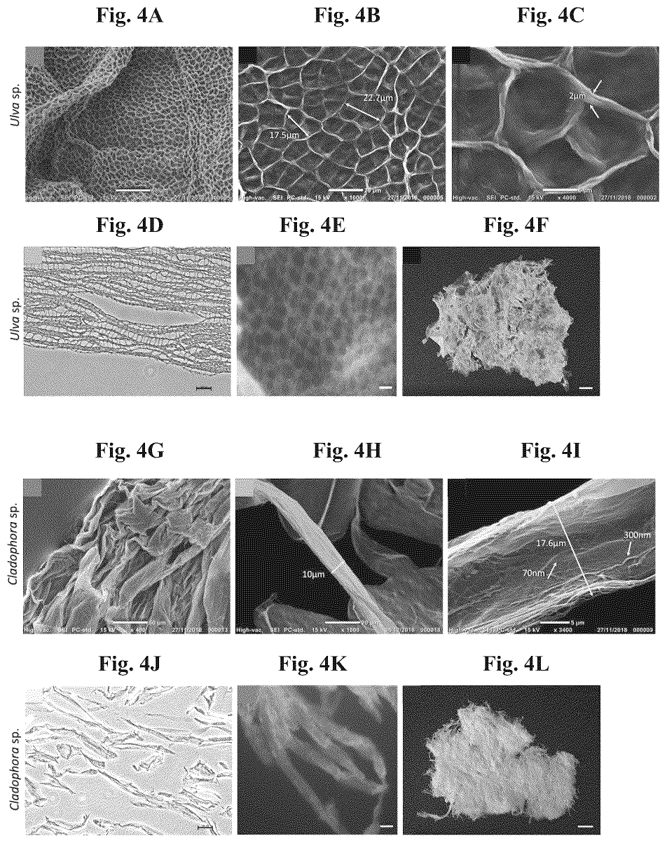

Using a digital caliper (Holex), Ulva sp. and Cladophora sp. scaffolds, obtained by drying at 40° C. in an oven for 24-48 h, were measured for their thickness, 0.1 mm and 0.15 mm, respectively (FIGS. 4F, 4L), and for their area dimensions for each experiment. Decellularized samples with area dimensions that range between 1 and 2 mm² were used for observation imaging analysis. Scaffolds for the biocompatibility tests were fabricated with specific dimension area for the viability direct test (uniformed 2 mm² circles) and cytotoxicity indirect test (6 cm² per 1 ml) as described below. Samples post-decellularization treatment were analyzed using fluorescent microscopy observation with Calcofluor White staining, SEM observations, H&E staining and DNA quantification, as described below.

Cellulose determination. To determine the presence of cellulose in the decellularized scaffolds, fluorescence staining solution consisting of Calcofluor White reagent (Ref. 18909; Sigma-Aldrich), which binds to cellulose in the plant cell wall, and 10% potassium hydroxide (KOH) (Ref. P5958; Sigma-Aldrich) (1:1) was used. The Calcofluor White fluorescent dye solution was deposited directly onto the seaweed decellularized samples, which were placed onto glass slides. Fluorescence Microscopy was used to observe the samples. The Evans blue present in the stain, emits fluorescence at a wavelength of 395-415 nm and permits a rapid visualization of cellulose presence in the decellularized seaweed cell wall (FIGS. 4E, 4K).

Seaweed cellulose scaffold histology. To evaluate and analyze the decellularized SC scaffolds, Ulva sp. and Cladophora sp. fresh and decellularized samples were embedded in paraffin and sectioned into 4 μm thick slices perpendicular to the surface. The sections were mounted on glass slides (4 sections per slides), stained with hematoxylin and eosin (H&E) reagent (Patholab, IL) and visualized under an optical microscope (Nikon Eclipse TS2, Japan). All image processing was performed with ImageJ software (ImageJ v. 1.51, NIH).

DNA quantification. The evaluation of acellular scaffold, emptied from its cellular organelles post decellularization, were further determined using plant genomic DNA concentration and purification analysis (Thermo Scientific GeneJET #K0791). The concentration was measured with a NanoDrop spectrophotometer (ND-2000, Thermo Scientific), used for a quick and simple wavelength absorbance analysis. Fresh and decellularized Ulva sp. and Cladophora sp. samples were examined (n=3 for each sample). Wavelength absorbance of all samples (1 μl solvent) were compared with blank sample and purified DNA sample with a nucleic acid to protein (A260/280) indicator and ratio between 1.7 and 1.9. Furthermore, gel electrophoresis (Invitrogen, E-Gel, 1.2%) was used to confirm the results. Purified DNA samples (20 μl solvent) of fresh and decellularized scaffolds were analyzed and documented (ENDURO GDS, Labnet; Omega Fluor, software).

Cell culture. Mouse embryonic NIH-3T3 fibroblasts (passages 33-53) stably expressing GFP-actin (NIH3T3-GFP-actin) were cultured in DMEM growth medium (GM) consisted of Dulbecco's Modified Eagle Media-high glucose with glutamine (DMEM-HG), supplemented with 10% fetal bovine serum, 1% L-glutamine, 0.1% penicillin-streptomycin solution (50 units/ml penicillin, and 50 μg/ml streptomycin), 1% sodium pyruvate solution, and 1% non-essential amino acids (all from Biological Industries, IL), in the 37° C., 5% CO₂ incubator. The GM was changed twice a week. Seeding was induced when a confluence of 80% was reached.

Seaweed cellulose scaffold sterilization. Decellularized cellulose from Ulva sp. and Cladophora sp. species were sterilized and pretreated prior to experiments. Single SC samples were placed in individual wells and soaked in 70% ethanol (1 ml/well) overnight at room temperature, in a tissue culture flow hood. Samples were then washed in ultrapure water (UltraPure DNsese/RNase-Free, Biolab-Chemicals), three times, then soaked overnight (2 ml/well) at room temperature, in a tissue culture flow hood. Next, the samples were treated in PBS (Dulbecco's Phosphate Buffered Saline (−) calcium (−), magnesium, Biological Industries) (1 ml/well) and incubated overnight (37° C., 5% CO₂). Finally, the samples were treated in GM consisting of DMEM-HG (1 ml/well), and incubated overnight (37° C., 5% CO₂). Successively, the media was discarded, and the samples were dried in a tissue culture flow hood, before recellularization seeding took place.

Recellularization of seaweed cellulose. Following the decellularization treatment, acellular SC scaffolds were recellularized with NIH3T3-GFP-actin cell culture to evaluate in vitro cell growth over a period of time. For the observational analysis tests, non-uniformed sized sterilized samples of the SC scaffolds (1-2 mm² dimensions area) were placed into a new non-treated 24-well plate (SPL Life Sciences). Single samples were placed in individual wells. Following, 5 μl of cell suspension at concentrations of 5, 10, 20 and 40×10³ cells/μl were seeded onto each scaffold and incubated (37° C., 5% CO₂) for 3 h to allow for initial cell adhesion onto the scaffolds. Following the initial incubation, 1 ml GM consisting of DMEM-HG was added into each well and resume incubation. Growth media was changed every other day. Cells were observed on the SC scaffolds for up to 8 weeks before fixation with 4% formaldehyde (PFA, Biological Chemicals) took place. All experiments had three replicates. Positive controlled samples of cell and scaffold without cells, as well as controlled blank samples, were observed and analyzed for this study.

Analysis and characterization. Scanning electron microscopy (SEM) analysis. Decellularized and recellularized SC scaffolds were evaluated and analyzed using SEM (JCM-6000, JEOL, Life Sciences, Tel Aviv University). Samples before and after cell seeding were visualized and recorded at x50, x130, x400, x650, x1000, x1700, x4000, and x7000 magnification. SEM images of the SC scaffolds, recellularized with NIH3T3 cell culture, were taken four weeks post seeding. Pore size, cell wall width, fiber diameter and cell culture morphology were observed and determined using image analysis software ImageJ (ImageJ v. 1.51, NIH). To determine the Ulva sp. pore size, 50 regions of the interest (ROI) were identified in a given SEM image of the decellularized sample, 10 ROI were identified to determine the Ulva sp. cell wall thickness, 55 ROI were identified to determine Cladophora sp. fiber width, and 50 ROI were identified to determine Cladophora sp. microfibrils overlay width. Moreover, 40 ROI were identified to determine the average cell size on the Ulva sp. scaffold and 70 ROI were identified to determine the average cell size on the Cladophora sp. scaffold. The mean dimensions and standard deviation are reported.

Confocal analysis. Real-time monitoring of the cell culture took place with fluorescence confocal microscopy (ZEISS LSM 510META). Images at week 5-6 recorded the NIH3T3-GFP-actin filaments (Argon gas laser 488 nm) and detected the scaffold reflection signal (633 nm). Cell growth was observed and analyzed with Zen (ZEISS microscopy) microscopy image processing and Imaris (Oxford Instruments). Additional time-lapse imaging (20×) of cell growth on the Ulva sp. cellulose scaffolds at Day 32 and on the Cladophora sp. cellulose scaffolds at Day 40 took place.

Biocompatibility evaluation. Following ISO standard 10993-parts 5 and 12, direct and indirect extract methods were used to evaluate in vitro cytotoxicity of macroalgae cellulose-based scaffolds Ulva sp. and Cladophora sp. Direct contact test allows for cell seeding directly onto the SC scaffolds, while indirect contact test method was carried out with cell culture incubated in media extracts from the SC scaffolds. Samples were evaluated and analyzed for this study. The mean cell metabolic activity and standard deviation are reported for each test.

alamarBlue assay. alamarBlue (AB) assay (BioRad, Enco, IL) was used to study and monitor the 3T3 mammalian cell culture viability in the presence of SC based scaffolds over time, following the manufacturer's protocol. AB detects the level of oxidation-reduction (REDOX) during respiration, by detecting the alteration of resazurin, fluorescent blue indicator dye that undergoes colorimetric change into resorufin, fluorescent pink, in response to cellular metabolic reduction. Thus, the increase in AB fluorescence signal over time is used as an indicator of fibroblasts metabolic activity, which is correlated indirectly to cell viability, expressed in cell proliferation and overall cell growth. Following the AB assay, cells were incubated in a 96 well plate with 10% v/v AB solution (200 μl p/well). Successively, duplicates of 100 μl solution samples were carefully distributed into a new 96 well plate. The percentage reduction of the AB dye was measured using a spectrophotometer microplate reader (Thermo Scientific, Multiscan Go) at 570 nm and 600 nm absorbance wavelength. Results were recorded using Skanit Software.

Scaffold cytotoxicity: indirect contact test with alamarBlue assay. Cytotoxic evaluation of Ulva sp. and Cladophora sp. SC scaffolds took place following ISO 10993-12, Biocompatibility Testing of Medical Devices, sample preparation for the “most severe” surface-area to volume exposure (6 cm² per 1 ml surface area, <0.5 mm thickness). Accordingly, sterilized Ulva sp. and Cladophora sp. SC scaffolds were fabricated (weight: 0.3845 g and 0.3493 g, thickness: 0.2-0.35 mm and 0.25-0.30 mm, respectively) and incubated (37° C., 5% CO₂) in DMEM GM for 24 h on a shaker (20 rpm). Concurrently, fibroblasts at cell density of 10×10³ cells p/well, were seeded and incubated for 12 h in a 96 well plate. The following day, media was extracted from each scaffold and filtered with 0.22 μm filters, to avoid remaining scaffold fragments. Cells were then incubated with 100% and 30% concentrations of media extracts for 24 h. Subsequently, absorbance measurements were taken after 4 h of incubation with 10% AB solution. Cytotoxicity evaluation was performed before and after the treatment with the media extracts, at the initial state (t=0) and after 24, 48 and 72 h of incubation (t=24, 48, 72), for both test groups. Additional control groups, including cells cultured with regular media, blank media and 10% AB solution in media, as well as cytotoxic positive control of 70% Methanol in media (30 min incubation prior to evaluation), were observed and analyzed for this study. The difference in percentage reduction of AB absorption between treated and control samples for each of the SC samples, at each incubation period were calculated and analyzed using the AB percentage difference equation (BioRad):

$$Percentage difference = \frac{( O 2 \times A 1 ) - ( O 1 \times A 2 )}{( O 2 \times P 1 ) - ( O 1 - P 1 )} \times 100 , \qquad ( 1 )$$

where O1 and O2 represent the molar extinction coefficient (E) of the oxidized AB at 570 and 600 nm, respectively; A1 and A2 represent the absorbance of the test wells at 570 and 600 nm, respectively; and P1 and P2 represent the absorbance of positive growth control well (cells and AB solution but no test agent-0% extract) at 570 nm and 600 nm, respectively.

Cell viability: direct contact test with alamarBlue assay. Ulva sp. and Cladophora sp. cellulose scaffolds were cut into uniformed circles (0=2 mm) with a hole puncher device, sterilized and placed into a 96 well plate, a single scaffold disc per well. Since we are unfamiliar with the cell growth on SC scaffolds, we used different cell densities in order to calibrate and optimize cell proliferation. Thus, following the recellularization method, each scaffold was seeded at an initial cell density of 5, 10, 20 and 40×10³ cells/μl (n=3). Additionally, control groups, including scaffolds without cell culture for each SC sample, blank media and 10% AB solution (media and AB but no cells), were observed and analyzed for this study. Following a 24 h incubation (37° C., 5% CO₂), AB assay was used to evaluate the cell culture viability in the presence of macroalgae cellulose scaffolds for a period of 6 weeks. It is worth noting that this method does not assure 100% accuracy detecting only the viability of cells on the SC scaffold alone. Thus, in order to reduce the chance of cell growth on the bottom of the well-plates, the seeded SC samples were transferred to a non-treated 12 well plate for continuous growth. Absorbance was measured after 24 h of incubation (37° C., 5% CO₂), with 10% AB solution. Continuous monitoring of the AB signal percentage reduction was performed at established time points (t=1, 2, 4, 8, 11, 15, 19, 22, 25, 29, 32, 36, and 40 days). The difference in percentage reduction of AB absorption between treated and control samples at each cell density and incubation period, were calculated and analyzed using the AB absorbance percentage reduction equation (BioRad):

$$Percentage reduction = \frac{( O 2 \times A 1 ) - ( O 1 \times A 2 )}{( R 1 \times N 2 ) - ( R 2 - N 1 )} \times 100 , \qquad ( 2 )$$

where O1 and O2 represent the molar extinction coefficient (E) of the oxidized AB at 570 and 600 nm, respectively; A1 and A2 represent the absorbance of the test well at 570 and 600 nm, respectively; R1 and R2 represent the molar extinction coefficient (E) of reduced AB (pink) at 570 and 600 nm, respectively; and N1 and N2 represent the absorbance at 570 and 600 nm, respectively, of negative control well.

Cell growth model. A logistic growth model was fitted to the results from the viability direct contact tests using Eq. (3):

$$N = \frac{{KN}_{0}}{{N}_{0} + ( K - {N}_{0} ) {e}^{- rt}} , \qquad ( 3 )$$

where N is the predicted cell viability at time t, K is the cell viability carrying capacity of the scaffold, N₀ is the cell viability at time t₀ (all represented by percentage reduction of AB), r is the cell proliferation rate, and t (days) is the time since to.

Parameters were determined for each scaffold type and for each initial cell concentration. K was determined as the maximum measured percentage reduction. t₀ was chosen as the time from which consistent growth was measured and N₀ was determined as the percentage reduction at time t₀. r was determined by minimizing the RMSRE, calculated by Eq. (4), using the Microsoft Excel Office 365 solver:

$$RMSRE = \sqrt{\frac{{\sum }_{i = 1}^{n} {( \frac{{N}_{pv} - {N}_{m}}{{N}_{pv}} )}^{2}}{n}} , \qquad ( 4 )$$

where Nₚᵥ is the modeled cell viability at time t, Nₘ is the mean of measured cell viability at time t, and n is the number of measurement points.

Statistical analysis. All experiments were carried out with at least three replicates. Values are presented as the mean±standard deviation (SD), paired with a two-sample T-test coupled with Fischer's Combined Probability test. Correlations between morphological parameters were evaluated using Spearman's correlation tests. A value of p<0.05 was considered statistically significant.

### Results

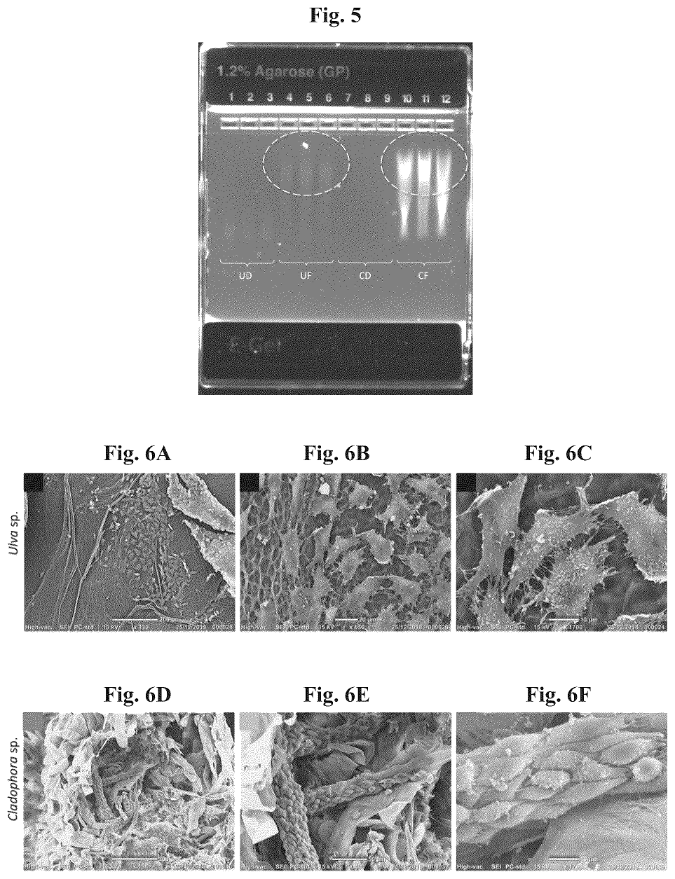

Seaweed decellularization. Fresh macroalgae species Ulva sp. (FIG. 1A) and Cladophora sp. (FIG. 1D) were obtained and examined for their structural composition variations, porous (FIG. 1B) and fibrous (FIG. 1E), respectively. Following, both species were decellularized to extract cellular content, obtaining a whole acellular natural seaweed scaffold (FIG. 2). Observation analysis, including scanning electron microscopy (SEM), fluorescent microscopy with Calcofluor White fluorescent dye that binds to cellulose, as well as histology analysis using hematoxylin and eosin (H&E) staining and DNA quantification test, were used to validate the decellularization treatment from both seaweed species, to determine cellulose explicit evidence, and to analyze both seaweeds structural composition variations (FIG. 4). SEM imaging of Ulva sp. (FIGS. 4A-4C) and Cladophora sp. (FIGS. 4G-4I) at different magnifications revealed no remaining of cellular organelles or nuclei content in either of the seaweed scaffolds. H&E imaging of the decellularized algae samples (FIGS. 4D, 4J) revealed the presence of eosin, which stained the cellular membrane in pink, and absence of hematoxylin, which stains cell nucleus in purple, in comparison to the H&E imaging of the fresh algae samples (FIGS. 1C, 1F), which reveals cell nucleus. This confirmed both seaweed matrices acellular, emptied from their cellular components. However, it is important to note that the eosin in the Ulva sp. sample (FIG. 4D) was shown to be more distinct than that in the Cladophora sp. sample (FIG. 4J). This could be due to the different cell membranes of the two seaweed samples and the fragmentation caused by the cross-section methods. Furthermore, DNA quantification analysis of the decellularized samples reveal low DNA concentrations for the Ulva sp. and Cladophora sp., with 5.53±2.80 ng/μl and 4.18±0.35 ng/μl concentrations, respectively, confirming acellular. However, it is important to note that although DNA concentrations of fresh algae samples were higher than the decellularized samples, they obtained overall low values (9.59±2.74 ng/μl and 69.74±16.50 ng/μl for Ulva sp. and the Cladophora sp., respectively), possibly due to the low DNA content extracted from the Ulva sp. sample compared to the Cladophora sp. sample. This was confirmed with gel electrophoresis analysis, which validated high DNA content for the Cladophora sp. sample, yet very blurry results for the fresh Ulva sp. samples (FIG. 5).

The decellularized seaweed samples were further verified for their cellulose content using Calcofluor White fluorescent dye, which allowed for direct visualization of the stained cell wall with fluorescent microscopy and confirmed the presence of cellulose as the prime structural component of both seaweed scaffolds (FIGS. 4E, 4K). All methods confirmed that the seaweed samples were acellular cellulose-based scaffolds, ready for use as ECM suitable for cell growth (FIGS. 4F, 4L).

Seaweed matrices structural characterization. Post decellularization, samples of Ulva sp. and the Cladophora sp. scaffolds (FIGS. 4F, 4L) were obtained for further analysis. Observation analysis confirmed the Ulva sp. and Cladophora sp. seaweed matrices' diverse structural compositions: porous and fibrous, respectively. Imaging of Ulva sp. matrix (FIGS. 4A-4E) revealed hollow cavities, organized in a comb-like network with highly interconnected pores, while imaging of Cladophora sp. matrix (FIGS. 4G-4K) revealed entangled mesh, bundled fibrillar matrix. These were evident in all observational assessments, including SEM, H&E staining, and Calcofluor White staining.

SEM imaging coupled with ImageJ software enabled structural analysis, and further understanding of the macroalgae acellular scaffolds' including shape, size and surface morphology. SEM imaging of the Ulva sp. and the Cladophora sp. scaffolds were taken at different magnifications (FIG. 4). The Ulva sp. matrix was observed to have interconnected cellulose web-like polygonal pattern, with uniform pore size average width of 20.2±4 μm dispersed along the matrix (FIG. 4B), and solid cell-wall ranging in width between 0.5 μm and up to 2 μm in the cell wall junctions (FIG. 4C), which confirmed a highly organized by-layer porous architecture and abundant surface area. In comparison, imaging of the Cladophora sp. scaffold revealed a highly packed, threadlike filamentous matrix, composed of heterogeneous fibers ranging in width from 5 μm and above 80 μm (FIGS. 4G-4I), overlaid with microfibrils ranging in width between 55 and 400 nm (FIG. 4I). However, due to the SEM metal coating we presume that the actual microfibrils diameters are even smaller. Furthermore, it should be noted that unlike the middle region shown with the SEM and Calcofluor White staining, the H&E staining, which reveals the cell wall membrane stained with eosin, shows cross-sections perpendicular slices. Thus, these images do not reflect the true structure of both algae samples. While the Ulva sp. show some full-size pores, in some areas, the Cladophora sp. is fragmented. Therefore, it was impossible to reveal the whole fibrous structure and clearly confirm the samples size or width based on these images.

Recellularization of seaweed cellulose scaffolds with mammalian cells. Observation analysis of the recellularized SC scaffolds enabled the evaluation of cell growth, cell morphology and biocompatibility. Readily sterilized scaffolds (1-2 mm²) were seeded with NIH3T3-GFP-actin fibroblast. The stable expression of actin-GFP by cells allowed us to follow the live cells cultured on the same scaffold at different time points from cell seeding and during the entire experiment.

SEM imaging analysis of both recellularized scaffolds, four weeks post seeding (FIG. 6), revealed a clear cell growth and cell attachments on the Ulva sp. porous scaffold (FIGS. 6A-6C) and on the Cladophora sp. fibrous scaffold (FIGS. 6D-6F). Imaging revealed Ulva sp. scaffold overlaid with viable cells that adhered onto the surface area in random directions. Cells stretched across individual cavities and spread onto the porous matrix surface, while others, adhered to neighboring cells and formed continues layers. Fibroblast reached an average cell size of 34.2±8.4 μm on the Ulva sp. porous scaffold (FIG. 6B). Additionally, imaging showed cells filaments protrusions aligned along the matrix cell-walls, utilize the cellulose lattice as a backbone platform for attachment sites. Otherwise, observations showed elongated thin protrusions that traced the matrix cell-wall ridges and juncture-sites, as well as cells that formed connectivity towards neighboring cells (FIG. 6C). Whereas SEM imaging of the Cladophora sp. fibrous scaffold exhibited cell attachments along the fiber's axis with elongated spindle-like shaped morphologies (FIG. 6E). Cells were observed to reach an average cell size of 20.1±4 μm on the Cladophora sp. fiber. The cells appeared to be fully attached to the scaffold's fibers. The cells long axis was aligned parallel to individual Cladophora sp. fibers, covering the fibers' surface area, with braid-like form, taking on the fibers' shape (FIG. 6F). Additionally, SEM imaging showed cells connectivity with other cells along the fibers. While some areas of both scaffolds are seen unpopulated by cells, these observations confirmed cell-to-matrix and cell-to-cell interactions on both SC scaffolds. However, further investigation of cell growth on both SC scaffolds should take place.

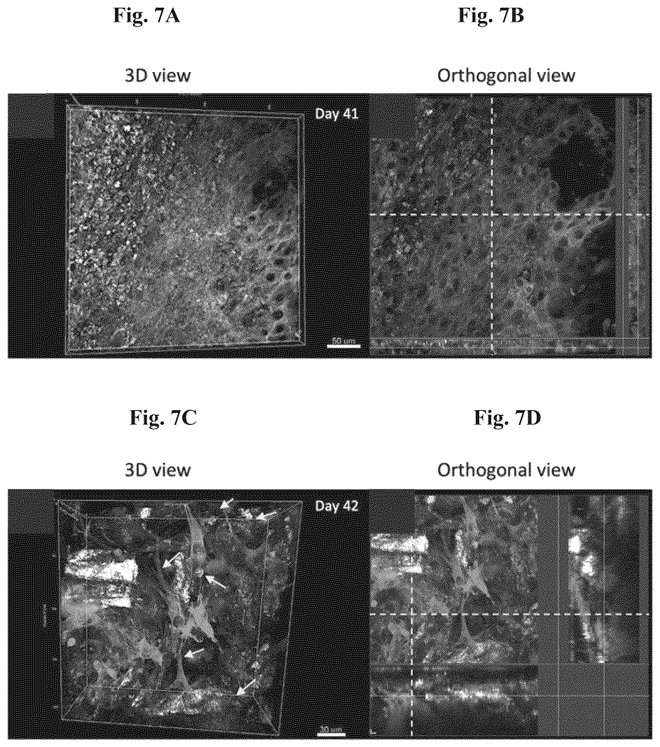

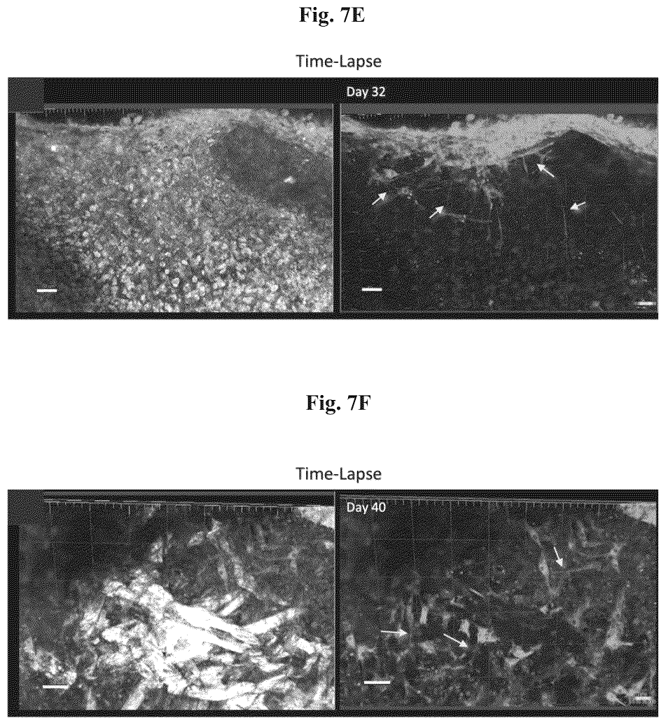

Confocal fluorescent imaging analysis of recellularized scaffolds enabled real-time monitoring and confirmed distinct cell growth, cell attachments and cell interactions onto both SC scaffolds. Shown here, 3D Z-stack and orthogonal confocal imaging of SC scaffolds Ulva sp. at day 41 (FIGS. 7A-7B) and Cladophora sp. at day 42 (FIGS. 7D-7E), recellularized with fibroblast (20×10³ cells/μl). The Ulva sp. scaffold's surface area appeared to be covered with confluent monolayer cell formation, demonstrating cell spreading onto the porous matrix surface (FIGS. 7A-7B). The Cladophora sp. scaffold showed cells attached onto individual fibers, with stretched morphologies typically elongated in the direction of the fibers. Cells were also observed to interspersed between the fibrous mesh and bridge between the fibers (FIGS. 7D-7E). Additionally, confocal imaging time-lapse of the Ulva sp. and Cladophora sp. scaffolds were taken at day 32 and 40, respectively, post recellularization (FIGS. 7C, 7F). The real-time imaging observations clearly show the formation of cell's long slender protrusions within the porous Ulva sp. network (>100 μm from nuclei center), and within the fibrous Cladophora sp. mesh, which verified cell spreading, attachments and migration within the SC scaffolds.

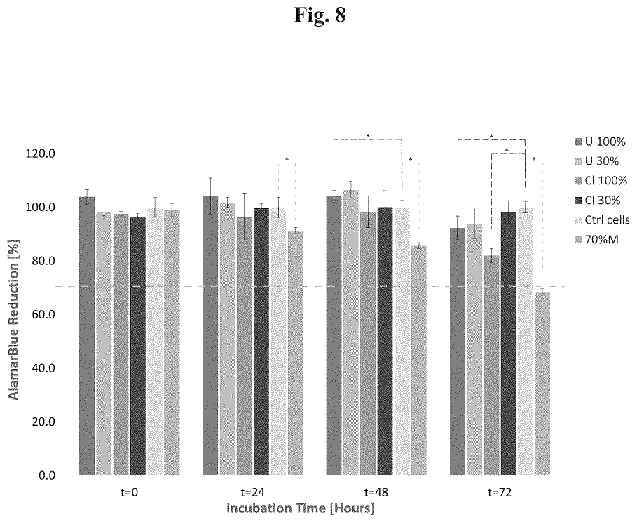

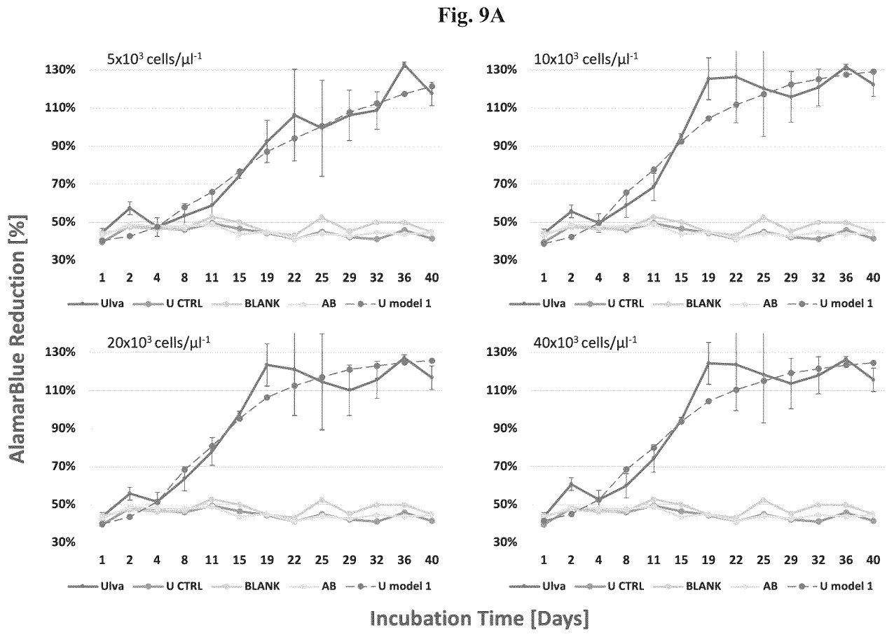

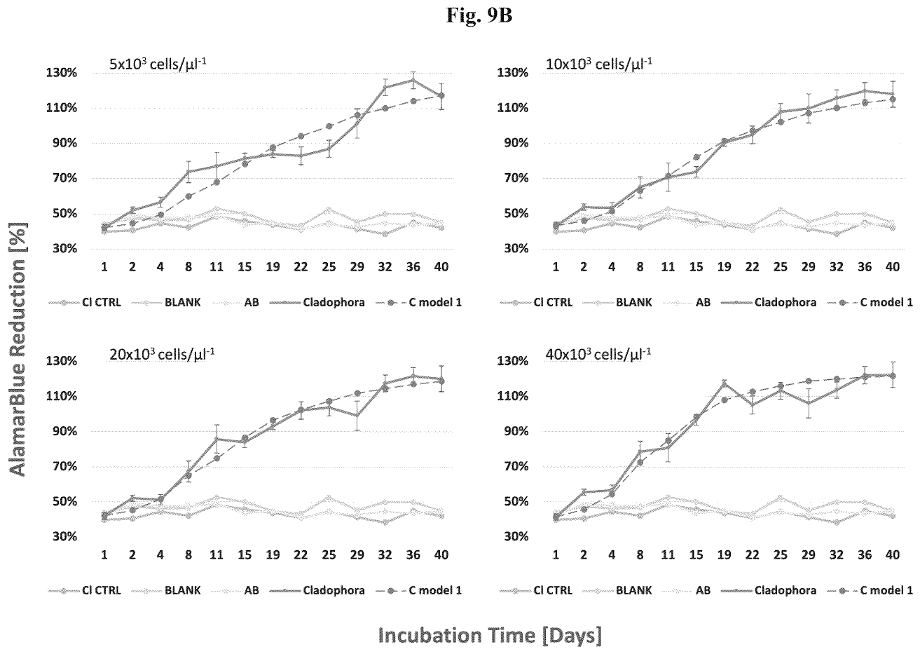

Biocompatibility of seaweed cellulose assessment with alamarBlue assay. AB colorimetric assay was used to evaluate the biocompatibility of the cellulose macroalgae scaffolds Ulva sp. and Cladophora sp. by means of quantitative assessment of cytotoxicity, and consequently cell proliferation, with both direct exposure to the scaffolds and indirect extract method, according to the international ISO-10993 standards 5 and 12 (ISO/EN10993-5; ISO/EN10993-12), used for biological evaluation of medical devices in animal testing and clinical trials. The main advantage of the AB method used is that it is non-toxic to cells and does not require fixation, which enabled us a continuous monitoring and evaluation of live cell viability over a long period of time without sacrificing the cells as required in other methods, such as MTT that is cytotoxic and could affect cellular morphology or cellular fate altogether. Results for cell viability and cytotoxicity for both SC scaffolds are shown in FIGS. 8-9.

Cytotoxicity evaluation of seaweed-cellulose scaffolds. The cytotoxicity for both cellulose-based macroalgae scaffolds was determined by the indirect media extract method, applied to fibroblasts cultured in cell-culture dishes. The relative change of AB fluorescence signal, which directly reflects the metabolic activity of the cell culture, was evaluated after 24, 48 and 72 h incubation with 30% and 100% media extracts concentrations (FIG. 8). AB absorbance measurements show similar cell growth at the start of the experiment (t=0) for all test and control groups (p>0.05), with minor variations due to the levels of cell coverage in each well. After 24, 48 and 72 h treatment, high AB reduction measurements above 80% were recorded for both the Ulva sp. and Cladophora sp. scaffolds, and for both 30% and 100% media extract. These results, showing more than the standard 70% viability, confirmed the non-toxicity of both SC scaffolds.

Cell viability evaluation with seaweed cellulose scaffolds. The AB assay enabled us to monitor live cell viability cultured on both SC scaffolds over a period of 40 days. The evaluation of cell growth with direct contact was determined by the relative increase of AB fluorescence signal over time, correlated to cell proliferation, in accordance with the AB assay, at four cell concentrations for each scaffold (FIG. 9). Results demonstrated that both the porous Ulva sp. and the fibrous Cladophora sp. matrices supported a long-term cell growth, indicated by an overall increase of AB reduction percentage with an average positive upward trend of 2.7-fold, with variation trends for both scaffolds. Cell viability on the Cladophora sp. scaffold showed a consistent and steady increase overtime (FIG. 9B), while cell growth on the Ulva sp. scaffold started with a steeper upward trend until week 3, followed by a stable plateau saturation level (FIG. 9A). However, it is important to note that although the seeded SC scaffolds were transferred to non-treated plates for the entire experiment, this method does not assure 100% accuracy detecting only the viability of cells on the SC scaffold alone.

Parametric Student's T-test comparisons coupled with Fischer Combined Probability test, show a highly significant difference (combined p<0.0001) between the Ulva sp. and Cladophora sp. scaffold test groups, for all four cell concentrations, as well as between the scaffolds' test and control groups. The viability results for all control-groups of the SC scaffolds without cells show no significant difference, with a stable AB percentage reduction mean of 45%±2.

More specifically, the plots at week one revealed a higher cell proliferation within the Cladophora sp. scaffold, with 71%±6.15 average percentage reduction for all cell densities, compared to 58.8%±4.18 for the Ulva sp., while cells on the Ulva sp. scaffold reached a higher proliferation from week 2 onwards (>90%±10.73) for all cell densities, compared to the Cladophora sp. scaffold (83.8%±9.5).

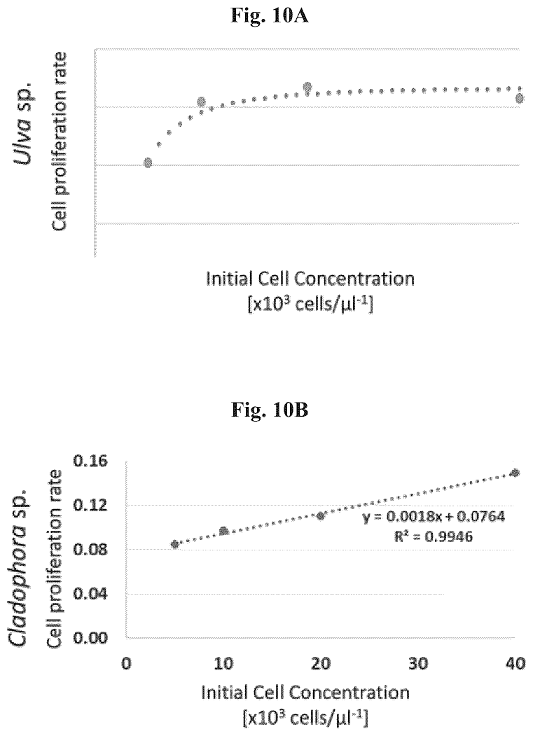

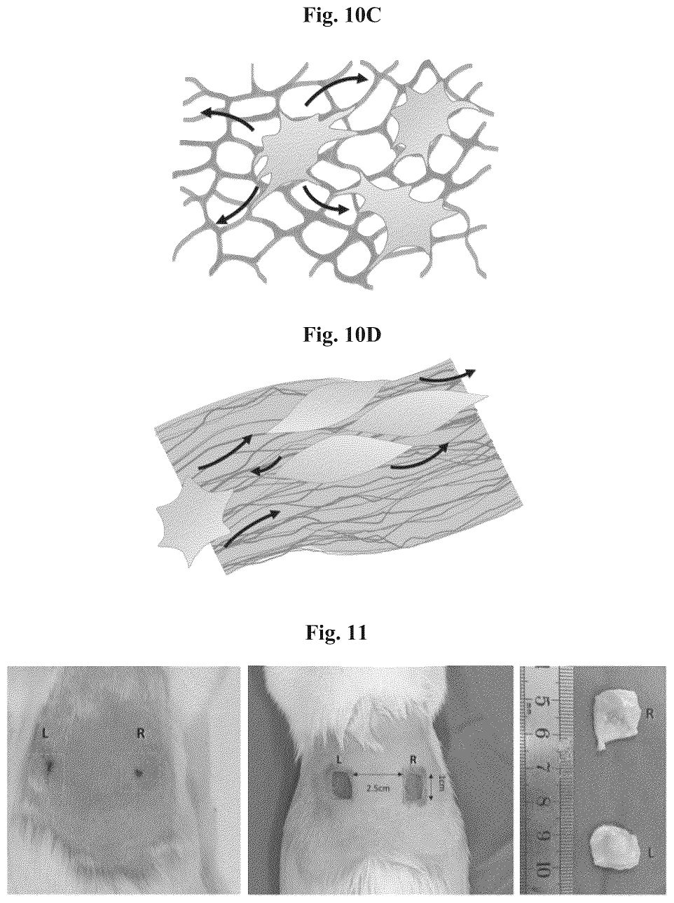

Cell proliferation rate increased in correlation to cell concentration. A logistic growth model, used to estimate cell proliferation rates in the different experiments, was fitted to the results from the viability tests, using Eq. (3). Cell proliferation rates (r) were calculated for each SC scaffold type and initial cell concentration (CO by fitting a proliferation model to data points of AB percentage reduction measured throughout the experiment. The prediction models, which obtained a root mean square relative error (RMSRE) of 0.077±0.007 for the Ulva sp. scaffold and 0.077±0.018 for the Cladophora sp. scaffold, were incorporated into FIG. 9 (dashed lines). Cell proliferation on the Ulva sp. scaffold was unstable during the first few days (lag period), therefore its to was set to the fifth day of the experiment (day 4). Cell proliferation on the Cladophora sp. scaffold was stable from the beginning, and thus its to was set to the time of the first measurement (day 1). Next, cell proliferation rates for both scaffold types were plotted as a function of initial cell concentration (FIGS. 10A-10B). Proliferation rate in the lowest initial cell concentration (5×10³ cells/μl) were similar for both scaffolds (r=0.08). However, the rate of cell proliferation on the Cladophora scaffold increased linearly with initial cell concentrations (R²=0.995), whereas the rate of cell proliferation on the Ulva scaffold, as a function of initial cell concentration, could be described as a second order Hill equation (r=0.134×Cᵢ²/(Cᵢ²+3.88²), RMSRE=0.043), leveling off at an initial cell concentration of 10×10³ cells/μl. In summary, the model exhibited that in the examined range initial cell concentrations affect proliferation rate differently on each SC scaffold type, following a second order Hill function on the Ulva sp. scaffold and a linear trend on the Cladophora sp. scaffold.

### DISCUSSION

The present study suggests novel cellulose scaffolds derived from marine green macroalgae species Ulva sp. and Cladophora sp. The SC scaffolds were extracted and analyzed for their structural variations and biocompatibility in vitro, and the structural-cellular interactions between the two SC scaffolds and NIH3T3 cells were examined.

Key considerations for selecting a suitable scaffold, when designing a bioartificial ECM environment, are its biocompatibility and ability to support cell growth and viability over time. Many natural and synthetic biomaterials are suitable resources for cell growth in tissue engineering. However, there is still an ongoing search for alternative, inexpensive matrices that could replace native tissue permanently. In recent years cellulose-based matrices have ignited novel bio-based scaffold fabrication (Hickey and Pelling, 2019; Modulevsky et al., 2014, 2016; Contessi Negrini et al., 2020). However, SC is still poorly investigated. Cellulose biopolymers from marine resources are attractive biomaterials, due to their little to none toxic reactions and natural antimicrobial bioactive compounds, relatively low cultivation and production cost as well as minor or absence of lignin content, and sustainable biostable features, which are appealing for applications that require no degradability and no conductivity as reinforcement, or as inert, composite biomaterials.

Decellularization could be achieved through numerous methods, including mechanical and enzymatic approaches. However, in order to achieve the best results to decellularize seaweed, while preserving structural composition intact, it was essential to fully decellularized a whole seaweed tissue from its cell content yet sustain undamaged cell wall. Following acid hydrolysis decellularization approach (Trivedi et al., 2016), and its optimization for a whole tissue sample, the removal of all cellular content from the macroalgae cell wall was achieved (FIG. 2). SEM imaging analysis were conducted to confirm the decellularization approach and to ensure that the acellular scaffolds maintained their core structure after the decellularization treatment. SEM imaging (FIGS. 4A-4F) of both seaweed matrices, confirmed an acellular, intact structural shape, obtaining the original tissue emptied from its cell content. Additionally, cellulose content was validated as the main cell-wall component for both SC scaffolds, Ulva sp. and Cladophora sp., with Calcofluor White fluorescent dye (FIGS. 4E, 4K), which has been proved to be an effective method for a simple and quick cellulose detection in plant tissues. These findings were consistent with previous studies of the two macroalgae species.

It is worth noting that utilizing strong chemicals for the removal of cell content and the isolation of cellulose has indeed proven effective; however, further optimization of the decellularization treatment is necessary to reduce or use no chemicals while promoting an economically and environmentally green approach. For example, pulsed electric field has been previously studied and shown to be effective, thus could be applied to decellularize SC, as well as sporulation inhibitors extraction that could further be explored to decellularize SC. Additionally, integrated process over direct cellulose extraction process can promote sustainable biorefinery design approach, for cellulose production with minimum environmental impact.

An additional key factor for selecting a suitable scaffold is its structural properties. On one hand scaffolds are required to advance cell growth, while providing structural and mechanical support for cell attachments on the ECM binding sites (Loh and Choong, 2013), and on the other hand they promote permeability to ensure the diffusion and transport of nutrient, cell signaling, oxygen, and growth factors, which in turn impact cell fate (Stevens and George, 2005).

Previous studies have shown direct correlation between scaffolds structural properties and cells behavior (Chang and Wang, 2011). In this study, macroalgae Ulva sp. and Cladophora sp. have demonstrated distinct cellulose variations: porous and fibrous, respectively. Thus, we hypothesized that variations of the SC scaffolds' structural morphologies, surface topographies and boundaries of the overall surface area (fiber width, porous tissue) enabled or limited cell attachments, cell spreading and migration orientations, and as a result influenced distinctly the fibroblasts cell growth, proliferation, and morphologies.

For example, in porous scaffolds, different pore size could directly promote or hinder cell functionality (Loh and Choong, 2013; Chang and Wang, 2011), thus ECMs with different pore sizes could be optimal for various tissue engineering applications (Chang and Wang, 2011). In comparison to other cellulose derived porous scaffolds, the Ulva sp. SC observed in this study consist of an intermediate pore size (10-30 μm) (FIG. 4B), which is larger than bacterial nanocellulose (BNC) pore size 1.66-98.7 nm (defined as the space between the BCN nanofibers), while smaller than terrestrial plant-based cellulose, e.g., apple, carrots and celery with pore sizes ranging between 70-420 μm (Contessi Negrini et al., 2020), and is also smaller than custom collagen sponges (50-200 μm), such as the BioMatrix (SpongeCol). Scaffolds with various pore size (50-350 μm) were described as macroporous, with pore sizes that exceed the cell size. Macroporous scaffolds are shown to promote cellular infiltration into the pores, and support adherence to the flat surface area around the cavities, or onto the pore walls, and thus increase 3D cellular organization. While microporous scaffolds with pore size (0.1-10 μm) that are smaller than the cell size limit cell invasion into the pores, and rather promote contact to the pore margins. Ultimately spreading onto the surface area, creating cell-to-cell interactions, and forming a continues sheet onto the scaffolds' surface area. For instance, MSC cells cultured on large pore size (>100 μm) displayed elongated stretched morphologies along the cell wall, while cells cultured on smaller pore size (<50 μm) displayed more oval-shaped morphologies with attachments in three-dimensions stretched across the pores. In comparison, fibroblast cultured on the Ulva sp. intermediate pore size scaffolds (FIG. 6A-6C) displayed polygonal stretched appearance, with cells size (34.2±8.4 μm) exceeding the average pore size (20.2±4 μm). SEM imaging revealed 2D cellular organizations of individual cells spread onto the SC surface, which initiated interactions with neighboring cells, while others formed monolayer ‘sheets’ onto the Ulva sp. surface area (FIG. 6A).

These findings are consistent with previous studies (Madub et al., 2021) and with the confocal imaging findings, conducted separately from the SEM imaging testing, here too the confocal imaging confirmed monolayer cell growth appearance (FIG. 7A). Moreover, the confocal imaging revealed elongated filaments protrusion that extended towards the matrix surface area, as well as through and in between the cavities, which were apparent in the GFP labeled actin stress fibers (FIGS. 7A, 7C), demonstrating cell-to-ECM interactions.

Consisting of high interconnected porous morphology and a distinctive intermediate pore size, we suggest that the Ulva sp. SC scaffold could provide a dynamic surface topography with abundant and evenly dispersed, attachment sites for continues cell growth, and spreading, and thus could impact cell migration directionality in more random orientation (FIG. 10C). These finding were consistent with previous studies of cell growth on flat 2D surfaces as well as 3D models with small porosity, which are characterized with flat and stretched monolayer morphologies, random growth directionality and good cell-surface interactions. Similar porous ECMs were also found to be advantageous for differentiation, cell proliferation, cell viability, and cell-cell and cell-ECM interactions, favorable to endothelial and dermal cells (Chang and Wang, 2011).

In comparison, fibers' properties in fibrous scaffolds, too were shown to have significant impact on cell fate. The Cladophora sp. observed in this study, obtain high fibrous matrix with a versatile fiber diameter (5-80 μm) (FIGS. 4G-4I), ranging from macroscale fibers that are found in plant cellulose, such as banana, sisal and coir (30-300 μm), to nanoscale microfibrils found in maze, cotton, celery and Arabidopsis thaliana (1-25 nm) (Rongpipi et al., 2019). Moreover, studies have shown that nanofibers enhance cell attachment and proliferation and effect cell spreading (Hsia et al., 2011; Chen et al., 2007). The microfibrils that overlay the Cladophora sp. fibers (FIG. 4I) were found to have a wide range of diameter width (40-400 nm), consistent with those found in nanocellulose derived from various sources, from BNC fibrils, (10-30 nm)), and lignocellulosic resources (<150 nm) to synthetic electrospun microfibrils (<500 nm) (Blakeney et al., 2011), fibrillar electrospun collagens (50-300 nm), and interestingly, in comparison with nontopographic grooved surfaces, ranging between 330 and 2100 nm, groove widths.

SEM imaging of fibroblasts cultured on Cladophora sp. scaffold displayed spindle-shaped elongated morphologies, with cell size (20.1±4 μm) smaller than the average fiber diameter (38.1±34 μm), and the cell's long axis appeared to be aligned parallel to the Cladophora sp. fibers (FIGS. 6D-6F). These growth patterns are consistent with cell morphologies found in native 3D fibrous tissue structures, as well as on topographical or grooved surfaces, which have demonstrated high influence on cell behavior, including the orientation, morphologies, and proliferation of cells by geometrical cues, associated with contact guidance (Bettinger et al., 2009). It is thus suggested that the high fibrillar surface topography, visible on the Cladophora sp. fibers (FIG. 4F), could affect contact guidance developments and therefore enhance cell attachments and elongated morphologies along the fibers, as well as guide cell spreading and migration directionality onto the fiber axis (FIGS. 6F, 10D).

Additionally, highly entangled matrices were shown to promote permeability, that advance cell survival, growth opportunities and cell attachments within the mesh layout, and bridge gaps between nearby fibers (Chen et al., 2007). Consisting of high entangled fibrous morphology, versatile fiber diameter and nanofibrils overlay, the Cladophora sp. SC scaffold in this study could provide with abundant topographical cues, for attachments and spreading along the fiber, and thus greatly contribute to the formation of connectivity between the cells as they attach onto the scaffold's fibers, and establish cell-fiber contacts, as well as cell-to-cell interactions, (FIGS. 6F, 7B), which impact cell growth, proliferation and cell migration orientation in one dimension (1D) along the fiber axis, as well as the formation of elongated filament protrusions between the fibers (FIG. 7D).

Similar to other cellulose derived biomaterials (Hickey and Pelling, 2019), these porous and fibrous SC models offer the necessary structural properties to support different cell types in numerous tissue engineering applications. For example, Ulva sp. intermediate pore size and Cladophora sp. fiber dimension could support mammalian dermal cells, and are suitable for drug testing, skin and wound healing applications (Loh and Choong, 2013. Thus, both seaweed structural properties could serve as an effective ECM when utilized as scaffolds for cell growth and have shown correlation to cell behavior with significant impact on cell morphology, attachments, and motility. It should be noted however, that cell growth and cell spreading in this study were shown to favor some areas of the scaffolds while evade other areas (FIGS. 6A, 6D), which could be attributed to the seeding technique. However, these findings, including cell dynamics and cell coverage on SC scaffold surface area, should further be investigated.

Another key consideration for selecting a suitable scaffold is biocompatibility, which ensures cell viability, proliferation, cellular attachments, and differentiation. In this study, the AB assay enabled both the monitoring of live cell viability, with direct contact test, over a long period of time without scarifying the cell culture, and the evaluation of cytotoxicity and cell viability with media extracts, indirect contact test. Both SC scaffolds demonstrated to be nontoxic, with 7.6% and 17.8% loss of metabolic activity, after 72 h incubation in 100% media extracts for the Ulva sp. and Cladophora sp., respectively (p<0.05), while maintaining a constant high viability in the presence of 30% media extract (p>0.05) (FIG. 8). Despite the reduction in cell viability, when exposed to 100% SC scaffolds media extracts for 72 h, cell viability above 70% is considered to be non-toxic in accordance with ISO 10993 standard, and was consistent with other studies. The cell viability decrease could be attributed to the adherence of protein from the media extract onto the SC scaffolds during incubation, as suggested in previous studies with collagen scaffolds.

In addition, cell viability analysis was evaluated through direct fibroblasts seeding, at various cell concentrations, onto the Ulva sp. and Cladophora sp. SC scaffolds. It should be noted that the SC matrices in this study were neither coated nor cross-linked with any additional reagents such as ECM proteins, which have been utilized in other studies, to enhance cell attachments prior to cell seeding (Modulevsky et al., 2014). Cell viability for both SC scaffolds and all four concentrations increased with an average positive upward trend of 2.7-fold during the experiment. These results are consistent with previous studies of viability tests that used AB with plant cellulose (Contessi Negrini et al., 2020) and marine collagen. Furthermore, the upward viability trends in this study showed a significant difference for the two SC scaffolds, with a combined p<0.0001 for all four cell concentrations.

However, differences in cell viability between the two scaffolds, and between cell concentrations, could be attributed to numerous reasons including cell growth rate correlated to initial cell seeding efficiency, matrices permeability, and exposure area, which impact cell fate opportunities. As well as, the SC scaffold structural properties (porous and fibrous), which offer advantages and disadvantages to cell growth and to cell-media-scaffold interactions, contact guidance, which orient cell attachments, and the overall shape of the scaffold, which provides boundaries for cell spreading and orientation. Thus, we propose that the two SC matrices structural properties and surface area that could be occupied by cells, provide a unique framework for cell growth, and therefore impact cell-to-cell interactions differently, which suggests the correlation between scaffold structural geometry and topography to cell fate and functionality. The Ulva sp. microporous scaffold enabled cell-to-cell interactions in all directions onto its surface area (FIGS. 6A-6C), advancing cell proliferation in all surface directions, in two dimensions (FIG. 10C). While, in comparison, cell spreading on the Cladophora sp. scaffold was limited by the fibers' width and guided by the overlaid microfibrils (FIGS. 6D-6F), advancing cell-to-cell interactions in one dimension, along the fiber elongated axis (FIG. 10D).

In addition to the observational analysis, cell proliferation onto the Cladophora sp. scaffold is supported also by the model, presenting a linear increase in proliferation rate as a function of initial cell seeding concentrations (FIG. 10B). Thus, it is hypothesized that the initial seeding concentration and the SC matrix structural surface area could determine the number of fibers along which proliferation occurs, and consequently impact the growth rate.

In contrast, cell proliferation on the Ulva sp. scaffold structural surface area (FIG. 10C) showed a slower proliferation rate in low concentrations, yet accelerated rapidly as concentrations increased (FIG. 10A). The Hill function, presented in this study, is commonly used to describe the relationship between the concentration of free ligand and the fraction of receptors bound by ligands. Thereby, we simulated the concentration of free ligands to free “migration opportunities”. Thus, in a two dimension structures we could obtain a second order Hill function in which cell growth has more “migration opportunities” in random directions, and as a result, the growth rate increases more rapidly, while cells ‘fill’ the scaffold's surface area, rates decreases due to saturation effects (FIGS. 10A, 10C). The structural features of each SC scaffold, differing cell alignments, facilitated cell migration, occupied the scaffold surface area in linear or all directions, which in return impact cell proliferation. Therefore, selective cell types on SC structures, could be highly advantage on the development of implanted devices (Loh and Choong, 2013; Chang and Wang, 2011).

### Study 2. In Vivo Studies

### Materials and Methods

Seaweed cellulose (SC) scaffolds preparation for in vivo. SC scaffolds were produced as described in Study 1. Briefly, fresh seaweeds, Ulva sp. and Cladophora sp., green marine macroalgae species, were collected and decellularized, through a 4-step sequential treatment with: 1. acetone (20% w/v, 60° C., 60 min) to remove pigments (chlorophyll) and proteins; 2. acetate buffer bath (sodium chlorite, 20% w/v, 60° C., 6-8 h), spurring bleaching and the removal of simpler structure polysaccharides; 3. 0.5 M sodium hydroxide bath (20% w/v, 60° C., 8-10 h) to remove all excessive lipids; and 4. 5% v/v hydrochloric acid (HCl) bath (20% w/v, 100° C., 10 min boil, following overnight at room temperature) to remove all excessive polysaccharides that might remain close to the cell wall. The SC samples were pH neutralized by washing repeatedly with distilled water (DW) between and after each step until reaching a neutral pH (SevenExcellence pH Meter). Finally, the samples were carefully rinsed (DW), and dried (room temperature). Next, Ulva sp. and Cladophora sp. SC scaffolds were fabricated using a hole puncher device, obtaining uniformed circles (diameter: Ø=8 mm; weight: 0.0042±0.0008 g and 0.006±0.0007 g; thickness: 2±0.06 mm and 1.7±0.05 mm, respectively). The scaffolds were then sterilized using 70% ethanol, following sterilization with a steam autoclave (121° C., 30 min), using sterilization pouches (YIPAK), the Ulva sp. and Cladophora sp. SC scaffolds were then ready to be used as porous and fibrous matrices for in vivo implantation.

Animal model. The surgical procedure and animal handling were performed at Tel Aviv University, in collaboration with Prof. Avshalom Shalom, Meir Medical Center (Israel). All experiments were approved by the Animal Care Committee of Tel Aviv University, conformed to the ethical guidelines for the use of animals in research according to the Institutional Animal Care and Use Committee (IACUC), as reflected in the Operational Guidelines for Ethics Committees for Biomedical Research.

Male Sprague-Dawley rats (approximately 250 g, n=18, 8 weeks old) were obtained from Envigo laboratories (Israel). The animals were housed in cages (3 animals per cage) with access to food and water ad libitum and were kept under a temperature and humidity-controlled room with a 12:12 hour light/dark cycle. All measures were taken to minimize the number of animals used, and to prevent pain and discomfort during the experiments.

Surgical procedure: SC scaffolds implantation. Subcutaneous dorsal surgery procedure was performed on all animals, including control groups without implants and experimental test groups with SC scaffold implantation, Ulva sp. (on the right side) and Cladophora sp. (on the left side), to evaluate their in vivo biocompatibility.

Before the implantation procedure, all animals were co-administrated with ketamine/xylazine (Clorketam/Sedaxylan, Phibro) anesthesia combination, and confirmed complete anesthesia by eliciting no response to a tail/paw pinch induced nociception stimulation.

An ophthalmic ointment was applied to prevent corneal irritation and drying. The back of each rat was shaved using an electric clipper. The exposed shaved skin area was disinfected with povidone iodine (Vitamed). Following, isoflurane anesthetic inhaler (USP 100%; Terrell, Piramal) was given, to maintain sedation for the remainder of the procedures, while animal hydration, body temperature and breathing were monitored.

Under anesthesia, two symmetrical and parallel full-thickness skin incision (1 cm each, right and left, at 2.5 cm from each other), were cut with a sterile scalpel along the upper dorsal area of each rat. Additionally, forceps were used to create small subcutaneous pouches for each implant. Next, the two SC scaffolds, Ulva sp. and Cladophora sp., were implanted into the right (R) and left (L) subcutaneous pouches, respectively. For all control group animals (n=9), no scaffold implantations were performed.

The incisions for all rats were then sutured (4/0 monofilament nylon polyamide, Atlas Medical) and the surgical sites were sterilized to prevent infection. Following, the rats were administered for analgesia (Rheumocam solution) and observed for healing in the following days. The experimental design is summarized in Table 1.

TABLE 1
In vivo Experimental design

| Model | Surgery | Tissue sample collection | | | |
|---|---|---|---|---|---|
| Treatment | Implantation (n = 2 sites) | Tissue with scaffold | | | Total observation |
| | per animal | (n = 2 sites) per animal: | | | period |
| | | Ulva sp. (right side) and | | | |
| | | Cladophora sp. (left side) | | | |
| Control | Wound w/no implants | Tissue (n = 2 sites) per animal | | | |
| | (n = 2 sites) per animal | (Right side and Left side) | | | |
| No. of replicates | n = 24 | n = 3 | n = 3 | n = 4 | (4 animals perished |
| per group | | | | | during experiment) |
| Animal group | Control | 1 | 2 | 3-4 | |
| number | Implant | 5 | 8 | 6-7 | |
| Time points (weeks) | 0 | 1 | 4 | 8 | 8 weeks |

Scaffold and tissue resections. At week 1, 4 and 8 post implantation, rats were euthanized using carbon dioxide (CO₂) overdose for tissue sample and implants collection (n=3 rats for each group; test and control). The dorsal skin surgical sites (n=2, left and right), for all animals, with and without implants, were carefully dissected (approximately 1-2 cm² sq), collected from each rat, documented, and immediately fixed with 4% paraformaldehyde (PFA, Biological Chemicals) for histological analysis (FIG. 11).

Histological analysis. Tissue samples, with and without scaffold implants, were embedded in paraffin blocks, sectioned at 5 μm thickness and mounted on glass slides (approximately 2-3 tissue sections from each block). All samples were cut through the center of the wound along the line perpendicular to the head-to-tail axis of the animal. Samples with implants were cut perpendicular to the scaffold.

Tissue sections were processed and stained with Hematoxylin-Eosin (H&E) and Masson's trichrome (MT) staining, by Patho-Lab Diagnostics Ltd (Rehovot, Israel) for the evaluation of cell infiltration and extracellular matrix deposition. Color images of each entire tissue section were acquired using NanoZoomer digital pathology slide scanner with 40× objective and NDP.view2 image viewing software (Hamamatsu, Hamamatsu City, Japan). Additional tissue sections from week 8 were processed and stained by Patho-Logica (Rehovot, Israel) with Anti-CD31/PECAM-1 immunohistochemistry staining, for the evaluation of vascularization (angiogenesis). Color images of each tissue section were acquired using KF-FL-400 digital pathology slide scanner with 40× objective and K-Viewer software (KFBIO, China).

Histopathological and biocompatibility examination. Slides were evaluated by a board-certified toxicological pathologist, according to the international ISO-10993 standards 6 used for the biological evaluation of medical devices in animal testing and clinical trials. Samples were examined in a “blinded” manner, i.e., without prior knowledge of the treatment groups. Histopathological evaluation was based on grades given to tissue tolerance parameters (e.g., inflammation, necrosis, foreign body response (FBR), fibrosis and neovascularization) according to the Harmonization of nomenclature and diagnostic criteria UNHAND) standards (https://www.toxpath.rg/inhand.asp#pubg). histopathological changes were described and scored by the Study Pathologist, using semi-quantitative grading of a five-point grading scale (0-4), taking into consideration the severity of the changes (0=no change, 1=minimal change, 2=mild change, 3=moderate change, 4=severe change), based on the criteria published by Schafer et al., 2018. The scoring reflected the predominant degree of the specific lesion seen in the entire field of the histology section.

criteria for adversity. The study pathologist included in the assessment appropriate judgment and conclusive statement concerning potential adversity/or not adversity for each type of treatment-related lesion. The criteria for adversity are based on the position papers published by the Society of Toxicologic Pathology (STP) and European ESTP (Kerlin et al., 2016; Palazzi et al., 2016). This statement refers to the animal species used in the experimental conditions specific to this study and will help the study director to determine the No Observed Adverse Effect (NOAEL) level (and Pass/Fail, in case of need). Parameters that may be taken into consideration for the determination of adversity include the presence of ulceration, necrosis, mineralization, and thrombosis, and potential recovery, according to the phases included in the study design (Baldrick et al., 2020). In particular, the severity grade and extension of such potential adverse lesion will be considered (Schafer et al. 2018). In case such listed lesion will be focal and of minor grade (up to grade 2 of 4), the lesion will potentially be considered as not adverse. However, in case such a lesion will be extensive, and of a higher grade than 2, such lesion may be considered adverse. In any case, the determination of adversity should always be analyzed and considered case-by-case, and the rationale for the suggested adversity should be justified with appropriate references.

Statistical analysis. All experiments were standardized; animals of the same age were used. We used at least three wounds from different animals for each treatment option. At least nine wounds (n=2 sites) were used as untreated, no implants, control groups. All experiments were carried out with at least three replicates. Values are presented as the mean±standard deviation (SD), paired with a two-sample T-test coupled with Fischer's Combined Probability test. A value of p<0.05 was considered statistically significant.

### Results

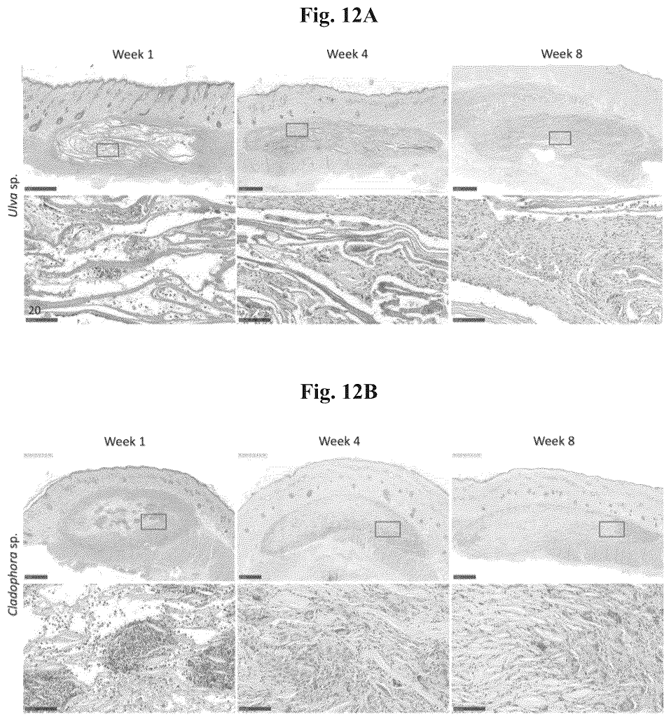

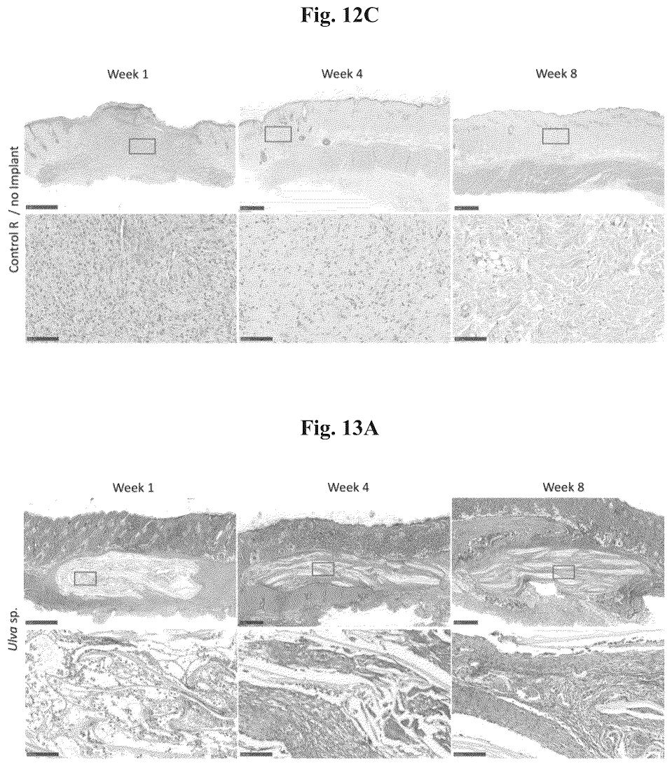

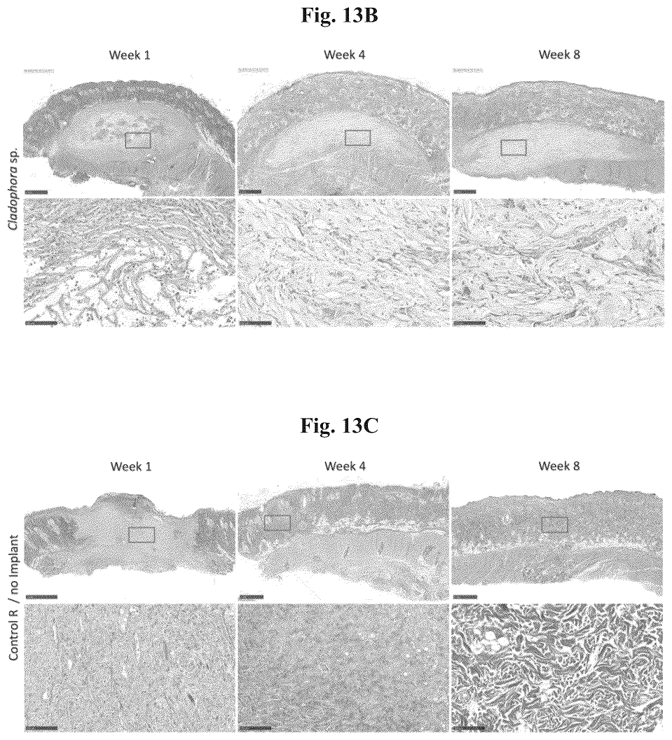

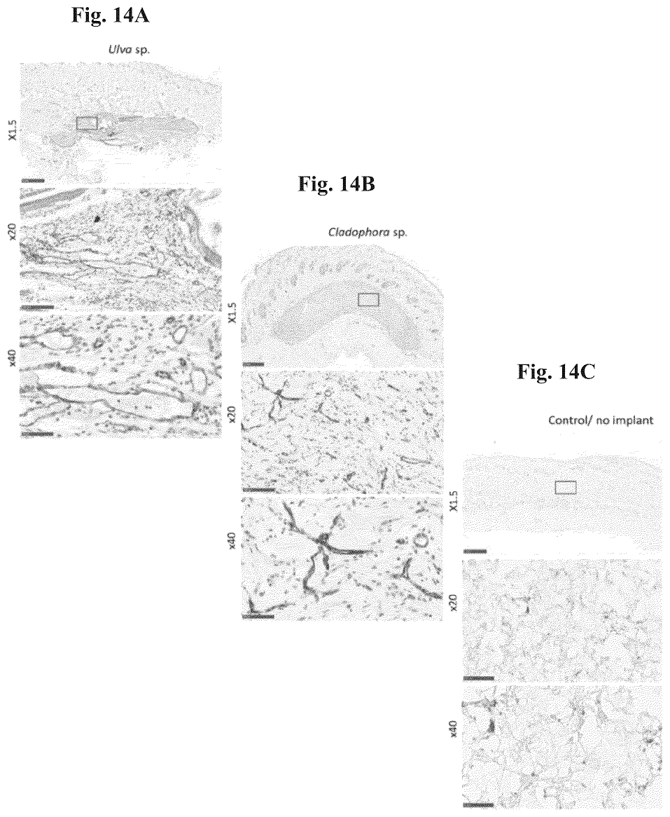

In this study, seaweed Cellulose (SC) scaffold from two marine macroalgae species Ulva sp. and Cladophora sp. were selected for their structural variations, porous and fibrous respectively, and evaluated for their in vivo biocompatibility as an alternative extracellular matrix (ECM) for biomedical applications. Scaffolds (Ø=8 mm) were subcutaneous implanted, separately, and independently, into two upper dorsal incisions (left and tight) in Sprague-Dawley rat model (male, 8 weeks old) (FIG. 11). Subsequently, implant excision sites were collected (FIG. 11) at week 1, 4 and 8, and processed for further histopathological evaluations and scoring to evaluate the morphological, inflammation reaction (H&E staining) (FIG. 12) collagen depositions (Masson's Trichrome staining) (FIG. 13) and vascularization (anti-CD31 antibody staining) (FIG. 14) at the Ulva sp. (FIGS. 12A, 13A, and 14A) and Cladophora sp. (FIGS. 12B, 13B, and 14B) implant sites, as well as at the control scar tissue with no implant (FIGS. 12C, 13C, and 14C). Moreover, the two SC implants were evaluated for their distinct structural impact on the healing process. Histopathological analysis by a Board-Certified pathologist revealed that in both SC implants, the nature of the reaction at week 1 post implantation was inflammatory reaction of foreign body reaction (FBR) nature. However, both SC implants undergo progressive fibrosis, gradually replacing the inflammation-FBR reaction by week 8. Furthermore, neovascularization formations were visible in both SC implant sites, throughout the 8-week study, indicating an ongoing healing process in both implants. Nevertheless, the overall healing process duration, formation, and reaction was significantly different at both SC implants. The fibrous Cladophora sp. implant site undergoes fibrosis formation from the center outwards to the periphery of the implant site (FIGS. 12B and 13B), accompanied by neovascularization, gradually replacing the center necrotic-inflammation reaction by week 8 (FIG. 14B), engulfing individual fibers with mature collagen depositions and FBR remainders at the periphery. In contrast, the porous Ulva sp. implant site undergoes fibrosis and neovascularization infiltration, intersperse between necrotic-FBR cavity collections that surround the implant fragments with mature connective tissue septa (FIGS. 12A, 13A and 14A). Both types of SC cellulosed scaffolds implants were confirmed to be locally nontoxic, and judged to be not adverse, according to the criteria of the Society of Toxicologic Pathology (STP). Moreover, the two scaffolds retain much of their original shape, providing biocompatible biomaterial that obtain intact permanent shape and form or long-term structural support for wound healing therapeutic, biomolecule release and tissue engineering applications.

### REFERENCES

Baldrick, P.; Cosenza, M. E.; Alapatt, T.; Bolon, B.; Rhodes, M.; Waterson, I., Toxicology paradise: sorting out adverse and non-adverse findings in animal toxicity studies. International Journal of Toxicology, 2020, 39(5), 365-378
Bettinger, C. J.; Langer, R.; Borenstein, J. T., Engineering substrate topography at the micro- and nanoscale to control cell function. Angew. Chem. Int., 2009, Ed. 48, 5406-5415
Blakeney, B. A.; Tambralli, A.; Anderson, J. M.; Andukuri, A.; Lim, D. J.; Dean, D. R.; Jun, H-W., Cell infiltration and growth in a low density, uncompressed three-dimensional electrospun nanofibrous scaffold. Biomaterials, 2011, 32, 1583-1590
Chang, H.-I.; Wang, Y., Cell responses to surface and architecture of tissue engineering scaffolds. In Regenerative Medicine and Tissue Engineering-Cells and Biomaterials (ed. Eberli, D.) 569-588 (IntechOpen, 2011)
Chen, M.; Patra, P. K.; Warner, S. B.; Bhowmick, S., Role of fiber diameter in adhesion and proliferation of NIH 3T3 fibroblast on electrospun polycaprolactone scaffolds. Tissue Eng., 2007, 13, 579-587
Contessi Negrini, N.; Toffoletto, N.; Fare, S.; Altomare, L., Plant tissues as 3D natural scaffolds for adipose, bone and tendon tissue regeneration. Front. Bioeng. Biotechnol., 2020, 8
Crapo, P. M.; Gilbert, T. W.; Badylak, S. F., An overview of tissue and whole organ decellularization processes. Biomaterials, 2011, 32, 3233-3243
Gibson, L. J., The hierarchical structure and mechanics of plant materials. J. R. Soc. Interface, 2012, 9, 2749-2766
Hickey, R. J.; Pelling, A. E., Cellulose biomaterials for tissue engineering. Front. Bioeng. Biotechnol., 2019, 7
Hsia, H. C.; Nair, M. R.; Mintz, R. C.; Corbett, S. A.m The fiber diameter of synthetic bioresorbable extracellular matrix influences human fibroblast morphology and fibronectin matrix assembly. Plast. Reconstr. Surg., 2011, 127, 2312-2320
ISO/EN10993-5. International Standard ISO 10993-5 Biological evaluation of medical devices: Tests for in vitro cytotoxicity. In Part 5: Tests for Cytotoxicity: In Vitro Methods, vol. 3, 42 (2009)
ISO/EN10993-12. International Standard ISO 10993-12 biological evaluation of medical devices: sample preparation and reference materials. In Part 12: Sample Preparation and Reference Materials (2012)
Kerlin, R.; Bolon, B.; Burkhardt, J.; Francke, S.; Greaves, P.; Meador, V.; Popp, J., Scientific and regulatory policy committee: recommended (“best”) practices for determining, communicating, and using adverse effect data from nonclinical studies. Toxicologic Pathology, 2016, 44(2), 147-162
Kumar, S.; Marrero-Berrios, I.; Kabat, M.; Berthiaume, F., Recent advances in the use of algal polysaccharides for skin wound healing. Curr. Pharm. Des., 2019, 25, 1236-1248
Loh, Q. L.; Choong, C., Three-dimensional scaffolds for tissue engineering applications: Role of porosity and pore size. Tissue Eng. B, 2013, 19, 485-502
Madub, K.; Goonoo, N.; Gimie, F.; Ait Arsa, I.; Schonherr, H.; Bhaw-Luximon, A., Green seaweeds ulvan-cellulose scaffolds enhance in vitro cell growth and in vivo angiogenesis for skin tissue engineering. Carbohydr. Polym., 2021, 251, 117025
Modulevsky, D. J.; Lefebvre, C.; Haase, K.; Al-Rekabi, Z.; Pelling, A. E., Apple derived cellulose scaffolds for 3D mammalian cell culture. PLoS ONE, 2014, 9, 97835
Modulevsky, D. J.; Cuerrier, C. M.; Pelling, A. E., Biocompatibility of subcutaneously implanted plant-derived cellulose biomaterials. Plos One, 2016, 11(6), e0157894
Nosar, M. N.; Salehi, M.; Ghorbani, S.; Beiranvand, S. P.; Goodarzi, A.; Azami, M., Characterization of wet-electrospun cellulose acetate based 3-dimensional scaffolds for skin tissue engineering applications: Influence of cellulose acetate concentration. Cellulose, 2016, 23, 3239-3248
Osorio, M.; Canas, A.; Puerta, J; Diaz, L.; Naranjo, T.; Ortiz, I.; Castro, C., Ex vivo and in vivo biocompatibility assessment (blood and tissue) of three-dimensional bacterial nanocellulose biomaterials for soft tissue implants. Sci. Rep., 2019, 9
Palazzi, X.; Burkhardt, J. E.; Caplain, H.; Dellarco, V.; Fant, P.; Foster, J. R.; Francke, S.; Germann, P.; Gröters, S.; Harada, T.; Harleman, J,; Inui, K.; Kaufmann, W.; Lenz, B.; Nagai, H.; Pohlmeyer-Esch, G.; Schulte, A.; Skydsgaard, M.; Tomlinson, L.; Wood, C. E.; Yoshida, M., Characterizing “adversity” of pathology findings in nonclinical toxicity studies: results from the 4^{th} ESTP International Expert Workshop. Toxicologic Pathology, 2016, 44(6), 810-824
Rongpipi, S.; Ye, D.; Gomez, E. D.; Gomez, E. W., Progress and opportunities in the characterization of cellulose—An important regulator of cell wall growth and mechanics Front. Plant Sci., 2019, 9, 1894
Schafer, K. A.; Eighmy, J.; Fikes. J. D.; Halpern, W. G.; Hukkanen, R. R.; Long, G. G.; Meseck, E. K.; Patrick, D. J.; Thibodeau, M. S.; Wood, C. E.; Francke, S., Use of severity grades to characterize histopathologic changes. Toxicologic Pathology, 2018, 46(3), 256-265
Stevens, M. M.; George, J. H., Exploring and engineering the cell surface interface. Sci. Rev., 2005, 310, 1135-1138
Trivedi, N.; Baghel, R. S.; Bothwell, J,; Gupta, V.; Reddy, C. R. K.; Lali, A. M.; Jha, B., An integrated process for the extraction of fuel and chemicals from marine macroalgal biomass. Sci. Rep., 2016, 6, 1-8
Wahlström, N.; Edlund, U.; Pavia, H.; Toth, G.; Jaworski, A.; Pell, A. J.; Choong, F. X.; Shirani, H.; Nilsson, K. P. R.; Richter-Dahlfors, A., Cellulose from the green macroalgae Ulva lactuca: Isolation, characterization, optotracing, and production of cellulose nanofibrils. Cellulose, 2020, 27, 3707-3725

What is claimed is:

1. A cellulose-based scaffold comprising a decellularized macroalgae tissue from which cellular materials and nucleic acids of the tissue are removed, wherein said scaffold has a fibrous structure.

2. The cellulose-based scaffold of claim 1, wherein said macroalgae tissue is a green macroalgae tissue, a red macroalgae tissue, or a brown macroalgae tissue.

3. The cellulose-based scaffold of claim 2, wherein said green macroalgae tissue is a Cladophora sp. tissue; and said red macroalgae tissue is a Bangia sp. tissue.

4. The cellulose-based scaffold of claim 1, wherein said macroalgae tissue is a Cladophora sp. tissue.

5. The cellulose-based scaffold of claim 1, wherein said fibrous structure has the form of a filamentous matrix.

6. The cellulose-based scaffold of claim 5, wherein said fibrous structure comprises heterogeneous fibers comprising fibers having a width of from about 0.5 μm to about 800 μm; microfibrils having a width of from about 0.55 nm to about 4 μm; and/or a combination thereof.

7. The cellulose-based scaffold of claim 1, wherein said cellulose-based scaffold is free of cellular organelles and nuclei content.

8. The cellulose-based scaffold of claim 1, wherein said fibrous structure is neither functionalized nor crosslinked.

9. The cellulose-based scaffold of claim 1, wherein said cellulose-based scaffold is free of non-biocompatible components.

10. The cellulose-based scaffold of claim 1, further comprising living animal cells adhered to said fibrous structure.

11. The cellulose-based scaffold of claim 10, wherein said living animal cells are mammalian cells.

12. The cellulose-based scaffold of claim 11, wherein said mammalian cells are selected from the group consisting of fibroblasts, myoblasts, umbilical vein endothelial cells (UVEC), and adipose mesenchymal stem cells.

13. The cellulose-based scaffold of claim 10, wherein said living animal cells have an average diameter of from about 10 μm to about 100 μm.

14. An implant comprising a cellulose-based scaffold according to claim 1.

15. The implant of claim 14, comprising living animal cells adhered to said fibrous structure.

16. The implant of claim 15, wherein said living animal cells are mammalian cells.

17. The implant of claim 16, wherein said mammalian cells are selected from the group consisting of fibroblasts, myoblasts, umbilical vein endothelial cells (UVEC), and adipose mesenchymal stem cells.

18. A process for the preparation of a cellulose-based scaffold comprising a decellularized macroalgae (seaweed) tissue from which cellular materials and nucleic acids of the tissue are removed, said process comprising the steps of:
(i) soaking a whole fresh macroalgae in an organic solvent, repeatedly for several times, to thereby remove pigments, lipids, and proteins;
(ii) washing the macroalgae biomass obtained in step (i) with water, and then bleaching with a sodium chlorite-containing buffer, to thereby remove polysaccharides other than cellulose;
(iii) washing the macroalgae biomass obtained in step (ii) with water to reach a neutral pH, and then performing an alkali treatment, to thereby remove lignin;
(iv) washing the macroalgae biomass obtained in step (iii) with water to reach a neutral pH, and then performing an inorganic acid treatment, to thereby remove excessive polysaccharides;
(v) washing the macroalgae biomass obtained in step (iv) in water to reach a neutral pH, to thereby obtain a clear cellulose biomass; and
(vi) optionally filtering and/or drying the cellulose biomass obtained in step (v) to thereby obtain said cellulose-based scaffold.

19. The process of claim 18, wherein
(a) step (i) is performed at a temperature ranging from room temperature to the boiling temperature of said solvent; and/or
(b) the bleaching in step (ii) is performed with an acetate buffer, at a temperature of about 60° C.; and/or
(c) the alkali treatment in step (iii) is performed with sodium hydroxide, at a temperature of about 60° C.; and/or
(d) the inorganic acid treatment in step (iv) is performed with hydrochloric acid, at a temperature of about 100° C.; and/or
(e) the macroalgae biomass obtained in one or more of steps (i)-(v) is stored under refrigerated conditions before being subjected to the next step; and/or
(f) the macroalgae biomass obtained in step (v) is filtered, and dried.

20. The process of claim 19, wherein
(a) said organic solvent in step (i) is acetone; and said step is performed at room temperature for at least few days, or at about 60° C. for about 60 minutes; and/or
(b) the macroalgae biomass obtained following said inorganic acid treatment in step (iv) is left at room temperature for several hours; and/or
(c) the macroalgae biomass obtained in step (v) is dried by freeze drying, at room temperature, or at a temperature above room temperature.

21. The process of claim 18, wherein said macroalgae is a green macroalgae.

22. The process of claim 21, wherein said green macroalgae is a Cladophora sp. or an Ulva sp.

23. The process of claim 22, wherein said green macroalgae is a Cladophora sp. and said scaffold has a fibrous structure.

24. The process of claim 22, wherein said green macroalgae is an Ulva sp. and said scaffold has a porous structure.

25. The process of claim 18, wherein said scaffold is neither functionalized nor crosslinked.

26. The process of claim 18, wherein said scaffold is free of non-biocompatible components.

27. The process of claim 18, further comprising the step of recellularization of said cellulose-based scaffold by implementing living animal cells on said cellulose-based scaffold.

28. The process of claim 27, wherein said living animal cells are mammalian cells.

29. The process of claim 28, wherein said mammalian cells are selected from the group consisting of fibroblasts, myoblasts, umbilical vein endothelial cells (UVEC), and adipose mesenchymal stem cells.

30. An implant comprising a cellulose-based scaffold obtained by the process of claim 18.
